(19) United States
(12) Patent Application Publication
(10) Pub. No.: US 2026/0117171 A1
(43) Pub. Date: Apr. 30, 2026

(54) Human Pregnancy on a Chip for Normal and Disease Biomarker Discovery and Preclinical Trials

(71) Applicants: Board of Regents, The University of Texas System, Austin, TX (US); THE TEXAS A&M UNIVERSITY SYSTEM, College Station, TX (US)

(72) Inventors: Ramkumar Menon, Galveston, TX (US); Lauren Richardson, Galveston, TX (US); Ananth Kumar Kammala, Galveston, TX (US); Enkhtuya Radnaa, Galveston, TX (US); Aum Han, College Station, TX (US)

(21) Appl. No.: 19/145,015

(22) PCT Filed: Jan. 10, 2024
(86) PCT No.: PCT/US2024/010978

§ 371 Date: Jul. 1, 2025

Related U.S. Application Data
(60) Provisional application No. 63/438,313, filed on Jan. 11, 2023.

(51) Int. Cl. C12N 5/073; C12M 1/00; C12M 1/12; C12M 3/00; C12M 3/06

(52) U.S. Cl. CPC C12N 5/0604; C12M 21/08; C12M 23/16; C12M 23/58; C12M 25/04

(57) ABSTRACT

Provided herein are devices and methods for mimicking the anatomy and physiology of a pregnancy comprising: a body defining a central cavity, inlet and outlet apertures in fluid communication with the central cavity of the body, wherein the central cavity comprises decidual cells; a first plurality of chambers in fluid communication with the central cavity, wherein the first plurality of fetal chambers comprise cells of fetal origin; a second plurality of chambers in fluid communication with the central cavity opposite the first plurality of chambers, wherein the first plurality of fetal chambers comprise cells of maternal origin; a third chamber in fluid communication with the central cavity, wherein the first plurality of fetal chambers comprise cells of the myometrium; wherein the integration of inputs and outputs from the central chamber, the first, second, and third pluralities of chambers mimics the anatomy and physiology of a pregnancy.

### CROSS-REFERENCE TO RELATED APPLICATIONS

This application claims priority to U.S. Provisional Application Ser. No. 63/438,313, filed Jan. 11, 2023, the entire contents of which are incorporated herein by reference.

### TECHNICAL FIELD OF THE INVENTION

The present invention relates in general to the field of the study of human pregnancy, and more particularly, to a novel human pregnancy-on-a-chip for the study of normal and diseased tissue, biomarker discovery, and preclinical trials.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with the study of human pregnancy.

Human pregnancy and parturition are fascinating but perplexing phenomena to comprehend. Two independent biological and physiological systems—fetal and maternal—that co-exist must be simultaneously considered to maintain pregnancy and aid in fetal growth and development. Parturition is a unique physiological process that reverses all homeostatic states of pregnant uterine tissues in a synchronized way to ensure timely and progressive delivery at term²⁻⁶. Preterm parturition (preterm birth [PTB], <37 weeks), a major complication impacting ˜12% of all pregnancies, contributes to 1 million neonatal deaths globally⁷⁻¹⁰. PTB is a syndrome initiated by failures in many of the Feto-Maternal uterine systems (F-M) that work together to maintain pregnancy, resulting in early initiation of labor and delivery^{1,11-15}. Neonates born preterm are predisposed to morbidities and mortalities and often have significant health issues throughout their lives¹⁶⁻²⁰. Mothers who deliver preterm are also at risk of postpartum complications^{12,17,18}. Therefore, reducing PTB risk is a global healthcare priority^{9,21}. Currently, it is thought that the mechanisms of delivery in normal parturition and PTB are different, but the true facts remain unclear^{22,23}. Reducing PTB risk remains a major challenge as this condition may arise with F-M medical indications or could be spontaneous with no known etiology^{1,6}.

Advances made in reproductive biology research have improved the knowledge of various F-M organs and their contribution to pregnancy and parturition, both term and preterm²⁴⁻²⁷. However, several challenges persist, which prevent making major impacts in reducing PTB¹⁶. It is necessary to better understand the coordinated inter-tissue interactions among various F-M uterine systems and their multiple functions that maintain a harmonious state of pregnancy. Although individual organ units (e.g., placenta, cervix, fetal membrane) are well-studied independently, understanding how they function cohesively as a system to maintain viable F-M integrity remains elusive. The role of resident immune cells and vaginal microbiome is also needed to understand normal pregnancy. It is also necessary to better understand the well-coordinated and timed event among the various systems that lead to homeostatic imbalances, triggering labor-associated changes that lead to delivery. Therefore, the inventors created a PTB model to understand disease development across various uterine tissues and signaling between various systems. By studying all F-M systems together, it is possible to determine signaling, communication mechanisms, and initiators/effectors of labor.

What is needed are systems and methods that allow for the interaction of the various tissue and cell types involved in pregnancy.

### SUMMARY OF THE INVENTION

As embodied and broadly described herein, an aspect of the present disclosure relates to a 3D microfluidic device mimicking an anatomy and physiology of a pregnancy comprising: a body defining a central cavity, an inlet aperture and an outlet aperture, wherein the apertures are in fluid communication with the central cavity of the body, wherein the central cavity comprises decidual cells; a first plurality of chambers in fluid communication with the central cavity, wherein the first plurality of fetal chambers comprise cells of fetal origin; a second plurality of chambers in fluid communication with the central cavity opposite the first plurality of chambers, wherein the first plurality of fetal chambers comprise cells of maternal origin; a third chamber in fluid communication with the central cavity, wherein the first plurality of maternal chambers comprise cells of the myometrium; wherein the integration of inputs and outputs from the central chamber, the first, second, and third pluralities of chambers mimics the anatomy and physiology of pregnancy. In one aspect, the inlet aperture and the outlet aperture allow for injection of at least one of fluid, gas and solid material within and through the device. In another aspect, the device is configured to be filled with cellular components in the first channel, second channel, top cavity and bottom cavity. In another aspect, the microfluidic device further comprises an outer surface for sealing the device to a support base. In another aspect, the microfluidic device further comprises a support base, optionally a glass support base. In another aspect, the device comprises a material selected from a group consisting of glass, silicon, polysiloxane, polydimethylsiloxane, and optically transparent polymers. In another aspect, the decidual cells form a decidual-placental interface, comprising one more cells selected from: decidua (DEC), syncytiotrophoblasts (STB), cytotrophoblast (CTB), and human umbilical vein endothelial cells (HUVEC). In another aspect, the fetal cells form Fetal membrane-decidual interface that comprise one or more cells selected from (4 cell types)—decidua (DEC), chorion trophoblast cells (CTC), extracellular matrix mesenchymal cells (MC), and amnion epithelial cells (AEC). In another aspect, the maternal cells for a Vagina-Cervix and comprise one or more cells selected from: cervical epithelial (ECTO and ENDO) and stromal cells (STR). In another aspect, the myometrium cells are selected from myometrial smooth muscle cells (MYO). In another aspect, the microfluidic device further comprises a fourth chamber comprising fetal brain cells (FB).

As embodied and broadly described herein, an aspect of the present disclosure relates to a lab-on-a-chip comprising a 3D microfluidic device mimicking an anatomy and physiology of a pregnancy comprising: a body defining a central cavity, an inlet aperture, and an outlet aperture, wherein the apertures are in fluid communication with the central cavity of the body, wherein the central cavity comprises cells of the decidua: a first plurality of chambers in fluid communication with the central cavity, wherein the first plurality of fetal chambers comprise cells of fetal origin; a second plurality of chambers in fluid communication with the central cavity opposite the first plurality of chambers, wherein the first plurality of fetal chambers comprise cells of maternal origin; and a third chamber in fluid communication with the central cavity, wherein the first plurality of maternal chambers comprise cells of the myometrium; wherein the integration of inputs and outputs from the central chamber, the first, second, and third pluralities of chambers mimics the anatomy and physiology of pregnancy. In one aspect, the inlet aperture and the outlet aperture allow for injection of at least one of fluid, gas and solid material within and through the device. In another aspect, the device comprises a material selected from a group consisting of glass, silicon, polysiloxane and optically transparent polymers, optionally wherein the polysiloxane is polydimethylsiloxane (PDMS). In another aspect, the width of the first channel is smaller than the width of the second channel. In another aspect, the lab-on-a-chip further comprises hydrogels contained separately within the first channel, second channel, bottom cavity and top cavity of the device. In another aspect, the second channel comprises a porous hydrogel of Type I collagen. In another aspect, the hydrogel of the top cavity comprises viable B cells, and the hydrogel of the bottom cavity comprises viable T cells. In another aspect, the top cavity further comprises chemokine CXCL13. In another aspect, the bottom cavity further comprises a chemokine selected from the group consisting of CXCL12, CCL21, and CCL19, or a combination thereof.

As embodied and broadly described herein, an aspect of the present disclosure relates to a kit comprising a lab-on-a-chip comprising: a 3D microfluidic device mimicking an anatomy and physiology of a pregnancy comprising: a body defining a central cavity, an inlet aperture and an outlet aperture, wherein the apertures are in fluid communication with the central cavity of the body, wherein the central cavity comprises cells of the decidua: a first plurality of chambers in fluid communication with the central cavity, wherein the first plurality of fetal chambers comprise cells of fetal origin; a second plurality of chambers in fluid communication with the central cavity opposite the first plurality of chambers, wherein the first plurality of fetal chambers comprise cells of maternal origin; and a third chamber in fluid communication with the central cavity, wherein the first plurality of maternal chambers comprise cells of the myometrium; wherein the integration of inputs and outputs from the central chamber, the first, second, and third pluralities of chambers mimics the anatomy and physiology of pregnancy; and one or more vials comprising the one or more cells of fetal and non-fetal origin.

### BRIEF DESCRIPTION OF THE DRAWINGS

For a more complete understanding of the features and advantages of the present invention, reference is now made to the detailed description of the invention along with the accompanying figures and in which:

FIG. 1 is an illustration that compares the organ structures of the intrauterine cavity to the present invention, Gravitas: The Pregnancy Chip, in which: AF—amniotic fluid, AEC—amnion epithelial cell, AMC—amnion mesenchymal cell, CTC—chorion trophoblast cell, DEC—decidua cells, HUVEC—human umbilical vascular endothelial cells, CTB—cytotrophoblasts, STB—syncytiotrophoblasts, MYO—myometrium, ENDO—endocervix, TZ—transformation zone, ECTO—ectocervix, MES—cervix mesenchymal cells, VEC—vaginal epithelial cells. This device concept can be adapted to contain dynamic flow in the DEC chamber to model maternal vasculature and interchange placenta trophoblast layers (i.e., with or without an extra villous trophoblast (EVT) layer) to model different trimesters.

FIGS. 2A to 2D documents the fabrication process of the Gravitas Pregnancy Chip. Three-step, photolithography was conducted to create microchannels connecting most cell chambers, followed by the second round of photolithography to create the pillar array for the TZ.

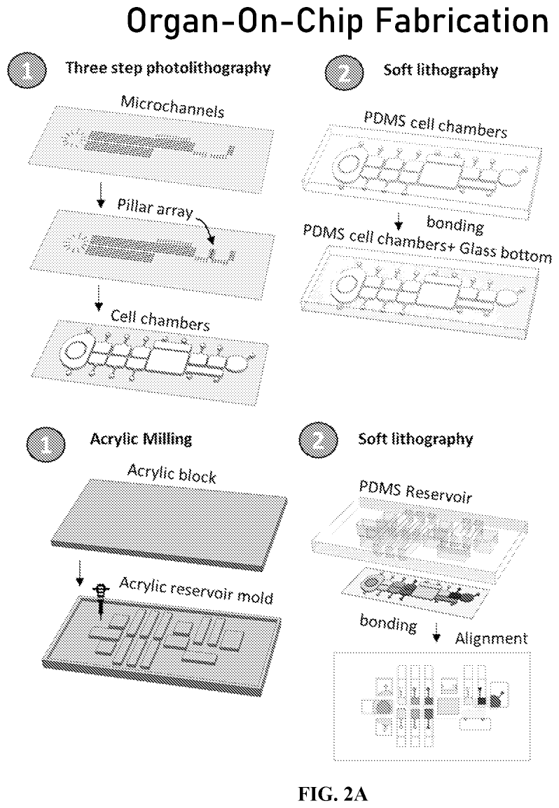

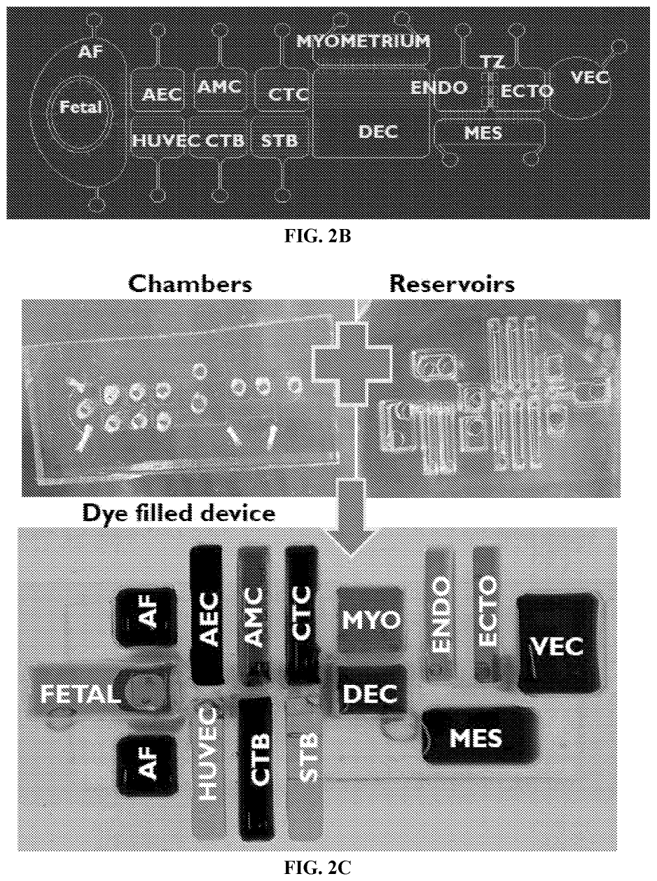

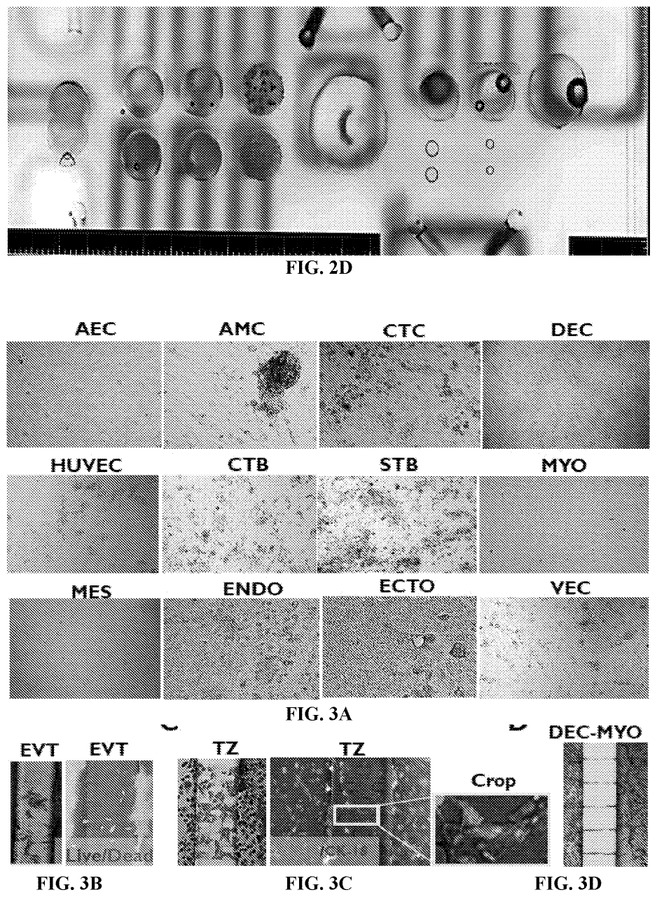

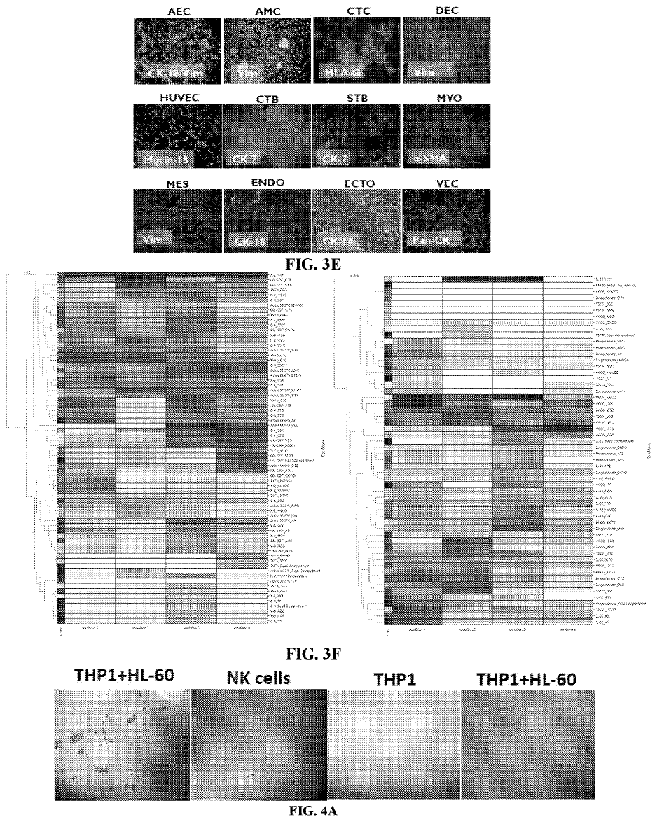

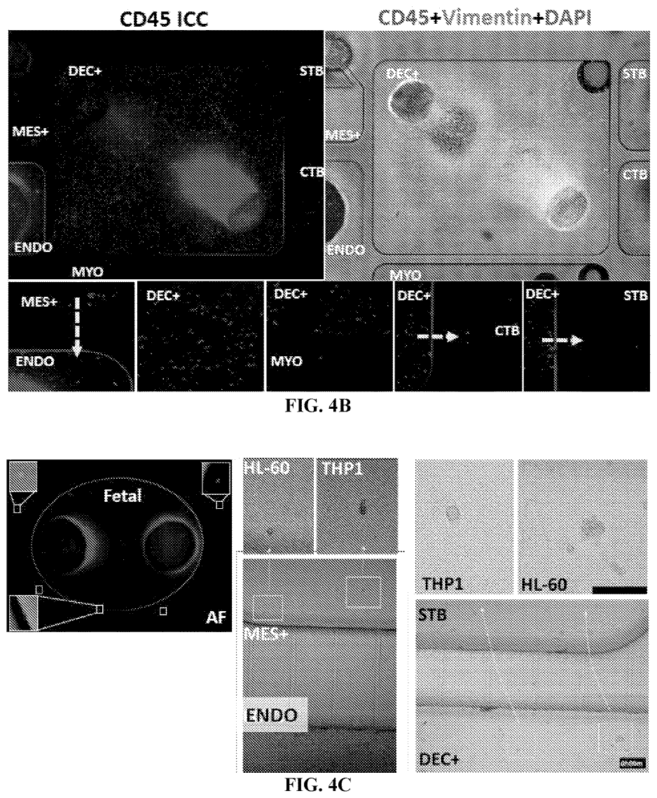

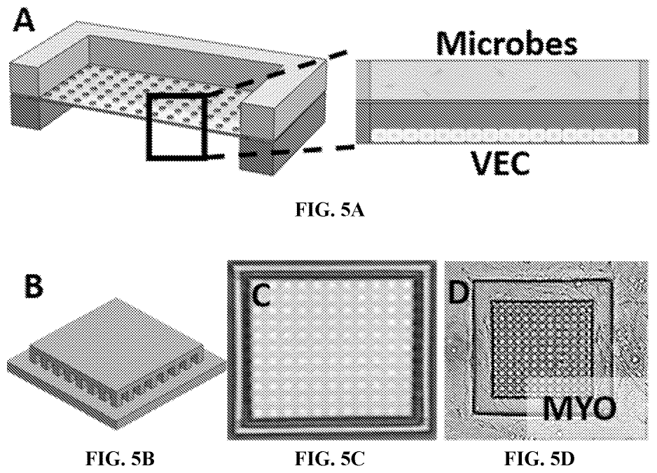

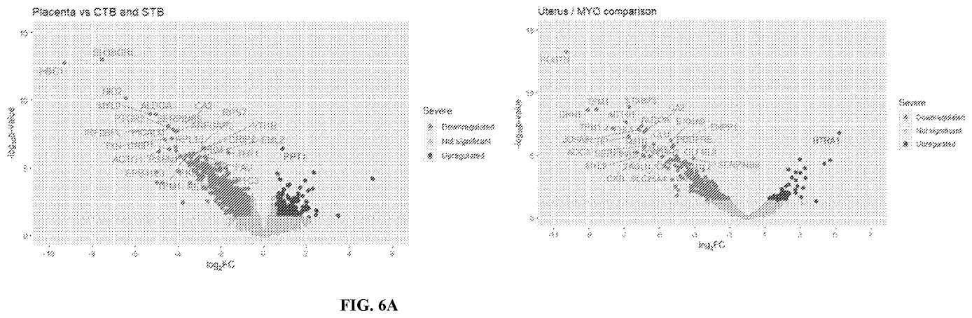

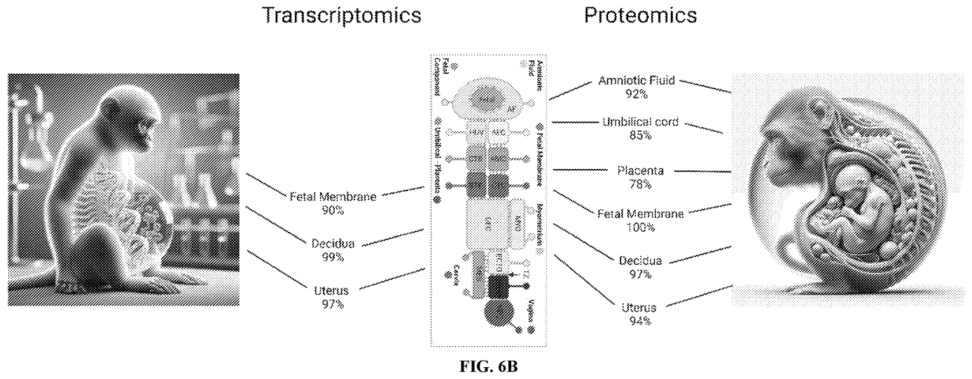

To complete the SU-8 master mold, cell chambers were added on top of the smaller structures (FIG. 2A). In the top Step 1, a three step photolithography is used to form microchannels, pillar arrays, and cell chambers are formed on a substrate. In top Step 2, soft photolithography is used to form the PDMS chambers are formed and bonded to the glass bottom/substrate from top Step 1. In bottom Step 1, acrylic milling is used to form an acrylic reservoir mold, which are used to make the PDMS reservoirs. In bottom Step 2, soft lithography is used for form the PDMS reservoirs, and the top and bottom are aligned and bonded. The images are shown as isometric views, with the bottom right being a top view showing the alignment of the reservoirs over the cell chambers.

Soft lithography utilizing polydimethylsiloxane (PDMS) was conducted to create the cell culture chambers that were bonded to a glass substrate, short from a top view (FIG. 2B). Master molds for the media reservoir were created using machine milling of acrylic blocks (FIG. 2C). Reservoir master molds were filled with PDMS to create media reservoirs for long term culture. PDMS media reservoirs were aligned to the inlets and outlets of the PDMS chamber chambers during the bonding phase to create the Gravitas: The Pregnancy chip device (FIG. 2D).

Microchannels are seen in red, the Transformation zone (TZ) pillar array in green, and cell chambers in white. FIG. 2B shows the PDMS cell culture chambers bonded to a glass slide and an example of the on-chip reservoir which is plasma bonded on top of the chambers (upper image), with the microfabricated prototype of the integrated model, where each cell culture chamber is filled with different color dye for easy visualization (lower image). Cell type is written in white. FIG. 3C shows brightfield image of the device showing cells after 3 days of culture. AF—amniotic fluid, AEC—amnion epithelial cell, AMC—amnion mesenchymal cell, CTC—chorion trophoblast cell, DEC—decidua cells, HUVEC—human umbilical vascular endothelial cells, CTB—cytotrophoblasts, STB—syncytiotrophoblasts, MYO—myometrium, ENDO—endocervix, TZ—transformation zone, ECTO—ectocervix, MES—cervix mesenchymal cells, VEC—vaginal epithelial cells.

FIGS. 3A to 3D show the Gravitas Pregnancy Chip characterization of cell-cell interactions and cell-specific markers. FIG. 3A: Brightfield images of cells within a Gravitas Pregnancy Chip after 72 hours. FIG. 3B: From left to right: Brightfield images showing EVT invasion into the pillar array separating the placenta trophoblasts from the maternal decidua. Calcein AM (live-green) and Ethidium bromide (dead-red) staining showing migrated cells inside pillar arrays are viable. This is a chamber that can be incorporated into this device to model the first and second trimester of pregnancy. FIG. 3C: Co-migration of Endo (cytokeratin [CK]-18) and Ectocervical (CK-14) cells into the pillar array forming the transformation zone (TZ) of the cervix. The TZ co-expresses both CK-14/CK-18. FIG. 4D: Microchannels separating the decidua (DEC) and myometrium (MYO) chambers show tight cell-cell interfaces. FIG. 3E: Fluorescent images of cells isolated from vaginal-cervix, fetal membrane, placenta, or myometrium tissue Immunostained for cell-specific markers within the POC. AEC—Vimentin (Vim) (green) CK-18 (red), AMC—Vim (green), CTC—HLA-G (red), DEC—Vim (green), HUVEC—Mucin-18 (green), CTB—CK-7 (red), STB—less expression of CK-7 than CYTO (red), MYO—α-smooth muscle actin (α-SMA) (red), MES—Vim (green), ECTO—CK-14 (green), ENDO—CK-18 (red), VEC—Pan-CK (red). FIG. 3F: Dendrogram representing overall pro-inflammatory (red) and overall anti-inflammatory (green) states of the pregnancy chip in different in vitro conditions (i.e., all cells, all cells+immune cells, all cells+immune cells+vaginal microbiome, all cells+vaginal microbiome). Secreted factors such as pro- and anti-inflammatory cytokines, growth factors, and hormones were measured from each chamber after 72 hours and mapped to identify clusters.

FIGS. 4A to 4C show immune cell characteristics within the Gravitas Pregnancy Chip. FIG. 4A: Brightfield images of cells chambers containing co-cultured immune cells within a Gravitas Pregnancy Chip after 72 hours. FIG. 4B: Fluorescent stitched images of the MES, ENDO, DEC, STB, and CTC chambers stained with DAPI (blue), Vimentin (green), and CD45 (red) to localize immune cells. Red CD45-stained immune cells predominantly stayed in resident DEC, MES, MYO, and Fetal chambers, while some cells crossed into neighboring chambers (yellow arrows or boxes). FIG. 4C: HL-60 neutrophils and THP1 macrophages were identified based on live cell videos by size, morphology, and movement.

FIGS. 5A to 5C show an on-chip microbiome chamber and contractile pillars. FIG. 5A shows a schematic of vaginal epithelial cell (VEC) and microbiome chamber separated by a semipermeable membrane. FIG. 5B shows an illustration of the flexible pad on which myometrium cells sit for easy contractility measurement. FIG. 5C shows a 3D printed PDMS microstructure. FIG. 5D shows myometrial (MYO) cells being cultured on top of the microstructure.

FIGS. 6A and 6B show differential proteomic analysis of non-human primate tissue compared to pregnancy chip derived cells after 72 hours in culture. FIG. 6A. The Log-fold changes and adjusted p-values for differential analysis of mass-spectrometry based proteomics profiles across pregnancy chip and NHP was calculated using moderated t-tests provided in the R package limma. Similar methods were applied for RNA-seq transcriptomics comparisons. Percentage of similarity between human pregnancy and pregnancy chip were recorded at each of the cellular, tissue and system levels at transcriptomic, metabolomic and proteomic domains. FIG. 6B. Summarizes the interactions corresponding to inter-cellular, inter-tissue and inter-system (fetal vs. maternal) levels at each omics domain will be studied individually for POC, human and NHP tissues using clustering with principal component analysis as well as network visualization methods. FIG. 6B. Statistical analysis was used to create a graphic image when the pregnancy chip was over 80% similar to nonhuman primate tissue in all of the organs.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Globally, preterm birth ((PTB); birth <37 weeks) impacts 15 million pregnancies and causes 1 million neonatal deaths annually. Reducing PTB requires a better understanding of the mechanisms of normal human pregnancy maintenance as well as initiation of parturition. However, the interplay between the various feto-maternal uterine tissues along with immune cells maintains a harmonious state for 40 weeks. Delivery of the fetus occurs when all the physiological balances collapse is a complex mechanism mediated by numerous factors that we do not yet understand well. Current in vitro and in vivo animal model studies have provided valuable information on respective organ systems' contributions to pregnancy maintenance and insights into malfunctions associated with PTB. However, since these various organ systems work together in pregnancy, reducing PTB rate, which has not dropped in the past 3 decades throughout the world, requires an approach that studies all systems together in a comprehensive and wholistic way.

The present invention is a “Pregnancy-On-a-Chip”, organ-on-a-chip (OOC), or Gravitas: The Pregnancy Chip, which replicates the multi-organ intrauterine system to study normal pregnancy, parturition, and PTB. The device has interconnected chambers with distinct maternal and fetal uterine compartments. One or more junctions between the chambers include decidual layers connecting the mother to the fetal membrane and the placenta. This OOC can be used to develop an integrated understanding of the multi-organ intercellular communication/signaling mechanisms underlying normal pregnancy and its disruption in PTB. This system can be used to evaluate and design novel therapeutics that restore the disrupted homeostasis underlying PTB. The interconnected OOC will use a combination of primary and immortalized parenchymal and immune cells, fetal cells and amniotic fluid component, as well as vaginal microbiome/metabolite components. The OOC system uses a straight forward design that incorporates advanced microfluidic features that allow easy experimental control over all the interconnected OOC sub-systems (applying localized stimuli and sampling fluids from each sub-system). This will allow for the establishment of one or more of the following: (1) a pregnancy model mimicking early various trimesters of pregnancy, (2) a normal parturition model that can determine the signaler for parturition (fetus, mother, or both), and/or (3) a model of infection or other disease states associated PTB that distinguishes this condition from normal pregnancy and parturition.

To address these challenges, the inventors developed an interconnected organ-on-a-chip (OOC) platform that integrates 5 key feto/maternal organ systems supporting pregnancy. These systems include placenta, fetal membrane, decidua, cervix, and myometrium, as well as representation of fetal components (e.g. amniotic fluid (AF), and in certain examples, brain). The skilled artisan will recognize that brain is an example here and can be interchanged with cells from any other fetal organs, e.g., heart, liver or lung. These 5 organ systems could be adapted (i.e., extracellular layers added such as the EVT or placenta stroma, or contain additional cell types such as immune cells) to model different stages of pregnancy. This system can encompass different cell types from F-M tissues, vaginal microbiome, and utero-placental immune cells to recreate a human “Pregnancy on a Chip.” This integrated model enables modeling normal pregnancy physiology and parturition phenotypes. This “healthy pregnancy” model can then be transformed into a disease model, which will replicate the infection-associated spontaneous PTB, which complicates ˜50% of PTB. Additionally, any risk factor exposed by the mother during pregnancy can be modeled (e.g., cigarette smoking, environmental pollutants, nutritional factors during pregnancy) and preclinical studies can be conducted to determine drug efficacity in delaying or preventing PTB. Development of fetal sex- and maternal race/ethnicity-specific cell lines further enhance the ability to decipher the impact of sex and race in health disparity associated with pregnancy, parturition, and PTB.

The present invention provides information for the Gravitas: The Pregnancy Chip (microfluidic organ on a chip (OOC) device) that can contain, multiple fetal and maternal cell types, from human pregnancy and covers the entire intra uterine cavity. The pregnancy on a chip use cell lines from different intrauterine tissues (e.g., 15-20 different cell types) and will be maintained in an environment like that seen in utero. The pregnancy on a chip device with cells and culture media, and other environments, is designed to mimic human pregnancy on a chip that can be used to study the interaction between the various tissues and/or cell type.

As used herein, the terms “integrated chip”, “microfluidic device”, and/or “organ on a chip (OOC)” are used interchangeably herein to refer to the Gravitas Pregnancy Chip taught herein, which is a microfluidic organ on a chip device that can include, e.g., multiple fetal and maternal cell types, from human pregnancy and covers the entire intra uterine cavity.

The chip is a microfluidic device that is interconnected using microchannels and maintains intercellular interactions. Therefore, this model represents a humanized model for various studies during pregnancy without using animal models that are not similar to a human pregnancy. As such, the pregnancy on a chip can mimic human pregnancy and be used for preclinical trials of drugs during pregnancy, which is currently difficult to test. It can also be used to reproduce various disease models in vitro by introducing one or more disease model cells and/or conditions. The pregnancy on a chip can also be used for biomarker discoveries to screen for high-risk pregnancies. Most importantly, this OOC device can be used for preclinical drug trials, toxicity testing of various toxicants and pollutants, as well as testing efficacy of drugs.

A microfluidic chip for use with the present invention will include a biocompatible substrate, one or more chambers disposed within the substrate, one or more microchannels between the chambers, and often a cover. As described herein, the substrate includes multiple chambers that include one or more openings (inlets and outlets) or channels that connect the chambers to allow fluid flow between the chambers. One or more openings are provided for loading cells and/or used to add or withdraw fluids and/or cells from the chambers.

As used herein, the term “substrate” denotes a variety of different biocompatible materials including polymeric materials, agent-binding surfaces, cell-binding surfaces, plastics, glass, or other manufacturable materials such as ceramic, metal, resin, gel, glass, silicon, glass-ceramics, and composites thereof. The substrate can be functionalized to, e.g., add a charge to the surface of the substrate to increase the adhesion of cells.

For cell and/or fluid delivery to and from a chamber or channel, the cell and/or fluid can be delivered via a top opening, via, a tube, or other suitable structure for transporting cells and/or fluids. The via or tube will be made from constructed from a biocompatible material, such as a polymer, plastic, glass, or other biocompatible material. Generally, each chamber will be in fluidic communication through a via or channel. Other types of fluidic connectors and/or reservoirs suitable for bringing fluidic communication (e.g., recreating blood flow through specific organs by incorporating dynamic flow) between chambers and/or the environment outside the device will be readily apparent to one of ordinary skill in the art.

Non-limiting examples of materials for use as a substrate with the present invention include, but are not limited to, polymeric materials such as polydimethylsiloxane (PDMS), polyurethane-methacrylate (PUMA), polymethylmethacrylate (PMMA), polyethylene, polyester (PET), polytetrafluoroethylene (PTFE), polycarbonate, parylene, polyvinyl chloride, fluoroethylpropylene, lexan, polystyrene, cyclic olefin copolymers, polyurethane, polyestercarbonate, polypropylene, polybutylene, polyacrylate, polycaprolactone, polyketone, polyphthalamide, polyacrylonitrile, polysulfone, epoxy polymers, thermoplastics, fluoropolymer, and polyvinylidene fluoride, polyamide, polyimide), cellulose acetate, fused silica, ceramic, glass (organic), metals, inorganic materials (glass, quartz, silicon, GaAs, silicon nitride), and/or other materials and combinations thereof.

The chambers and the microchannels connecting the same can be surface-modified chemically to enhance wetting or to assist in the adsorption of the tissues or cells. Non-limiting examples of surface-modifying chemicals can include, but are not limited to, silanes such as trimethylchlorosilane (TMCS), hexamethyldisilazane (HMDS), (Tridecafluoro-1,1,2,2-tetrahydrooctyl)trichlorosilane, chlorodimethyloctylsilane, Octadecyltrichlorosilane (OTS) or γ-methyacryloxypropyltrimethyoxy-silane; polymers such as acrylic acid, acrylamide, dimethylacrylamide (DMA), 2-hydroxyethyl acrylate, polyvinylalcohol (PVA), poly(vinylpyrrolidone (PVP), poly(ethylene imine) (PEI), Polyethylene glycol (PEG), epoxy poly(dimethylacrylamide (EPDMA), PEG-monomethoxyl acrylate; surfactants such as Pluronic surfactants, Poly(ethylene glycol)-based (PEG) surfactants, sodium dodecylsulfate (SDS) dodecyltrimethylammonium chloride (DTAC), cetyltriethylammonium bromide (CTAB), or Polybrene (PB); cellulose derivatives such as hydroxypropylcellulose (HPC), or hydroxypropylmethylcellulose (HPMC); amines such as ethylamine, diethylamine, triethylamine, or triethanolamine, fluorine-containing compounds such as those containing polytetrafluoroethylene (PTFE) or TEFLON®.

Adhesion-promoting materials for use with the present invention can include but are not limited to: poly-L-Lysine, poly-D-Lysine, high-molecular-weight cationic copolymer of polyacrylamide and quaternized cationic monomers, poly dopamine, collagen, fibronectin, fibrin, gelatin, poly gelatin, extracellular matrix (ECM) proteins or peptides, ECM-like proteins or peptides, and combinations thereof.

Generation of cell lines from F-M uterine tissues. The inventors developed readily accessible and stable primary cells and/or cell lines that phenotypically and functionally represent in utero systems. Genetically/morphologically well-characterized cell lines from primary tissues have already been established from each intrauterine system. Using these cells, it is possible to mimic 3^{rd} trimester pregnancy, as this period is highly vulnerable to both endogenous and exogenous factors^{1,21,28,29}. Two largely ignored areas in reproductive medicine are health disparities arising from fetal sex³⁰⁻³⁸ and maternal race contributing to PTB³⁹⁻⁵⁶.

TABLE 1
Cell lines and availability

| | |
|---|---|
| Cell type | Immortalization Method |

Cervical Compartment

| | |
|---|---|
| VEC | HPV16E6E7‡ |
| Ecto | HPV16E6E7‡ |
| TZ | Developed naturally on chip by Endo-Ecto |
| | migration |
| Endo | HPV16E6E7‡ |
| Stroma | hTERT‡ |

Fetal Membrane Compartment

| | |
|---|---|
| AEC | HPV16E6E7* |
| MC | HPV16E6E7* |
| CTC | SV40T* |
| DEC | HPV16E6E7* |

Placenta Compartment

| | |
|---|---|
| HUVEC | HPV16E6E7* |
| CTB | SV40T* |
| STB | Transitioned CTB by Forskolin |
| EVT | Natural formation from STB on chip |

Myometrium Compartment

| | |
|---|---|
| MYO | hTERT‡ |

Fetal brain compartment

| | |
|---|---|
| SVGp12 + HMC3 | SV40T‡‡ |

‡Available in our biobank;
*Available in biobank; sex and race-specific cells are prepared for personalized testing;
‡‡Available from commercial vendors

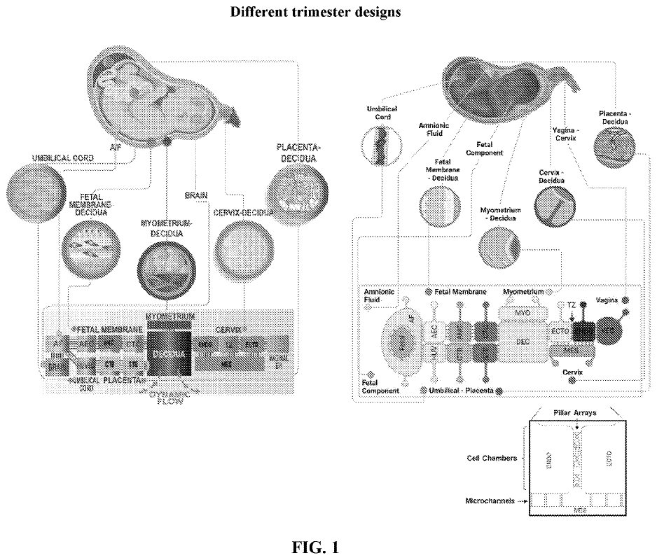

Pregnancy physiology is substantially influenced by fetal sex³⁰⁻³³ where pregnancies carrying a male ferns are more prone to PTB³³⁻³⁶. African Americans have a higher rate of PTB than Caucasians^{29, 39, 51-57-99}. Fetal gender- and maternal race/ethnicity-specific cells and cell lines are established from umbilical cord, fetal membranes, placenta, and decidua. The cells are immortalized and then characterized cells for phenotype, markers, and genetic stability. A schematic of the F-M system to be used is presented in FIG. 1.

Generation of cells: The Gravitas Pregnancy Chip can include all major uterine systems represented by multiple cell types from the pregnant uterus and the fetus itself. The DEC can include immune cells and can be represented in a single compartment. FB is obtained from vendors, already available, and can be fetal sex-matched in the experiments. Obtaining all tissue biopsies (vagina, cervix, and myometrium) needed to prepare respective cell lines from the same pregnancy is practically impossible, so cryopreserved maternal race-specific uterine tissues can be used to establish these cell lines.

Primary cells can be converted into immortalized cell lines using approaches outlined in Table 1. A biobank of immortalized cells can be used for all studies to limit subject-to-subject variabilities. Amniotic fluid (AF). The AF chamber will contain subject derived or commercially available synthetic or simulated sterile AF⁷²⁻⁷⁴. Biobanked AF samples from normal-term Cesareans available with investigators of this project can be used as an alternate approach.

Development of an integrated OOC platform that models the entire F-M uterine tissue. Several of the existing OOC models developed for placenta, amnion membrane, and fetal membrane-maternal interface⁷⁵⁻⁸³ are basic 2-cell-type co-culture models. However, a few that incorporate more than 2 from both fetal and maternal cells are emerging^{81,83}. Studies using these systems range from very simple drug perfusion studies to more complex ascending infection (from mother to fetus) studies^{81,84,85}. There is one very simplistic model of cervix⁸⁶, while none exists for myometrium. A major shortcoming of most of these models is that they are missing important cell types seen in utero (e.g., EVT, TZ, and cervical stroma; immune cells in decidua). Importantly, in utero these different F-M tissues work cohesively to maintain homeostasis, and interconnected OOC models are required to study these interactions.

Experimental Approaches. Gravitas: The Pregnancy Chip design: The interconnected OOC model will contain multiple fetal and maternal cell types co-cultured, representing 5 different pregnancy-related intrauterine organ systems and the fetus. There are broadly 2 different co-culture microfluidic chip designs, vertical and planar. The vertical design utilizes a top-bottom cell culture chamber separated by a porous membrane. Although such a design can mimic multiple layers of co-cultured cells, it is not possible to create a stack of more than 2-3 cell culture chambers and is also not compatible with easy imaging. A planar co-culture design utilizes cell culture chambers separated by arrays of microfluidic channels, where the channel array allows cell-cell communication as well as active cell migration. Since multiple culture chambers can be connected in series, co-culturing tens of different cell types is possible while being also compatible with direct microscopy; thus, observing cell growth, migration, and phenotyping is easy. FIG. 1 also shows the design, where 15-20 rectangular co-culture chambers are interconnected through arrays of microfluidic channels.

FIG. 1 is an illustration that compares the organ structures of the maternal-fetal interface and the Gravitas Pregnancy Chip in which: AF—amniotic fluid, AEC—amnion epithelial cell, AMC—amnion mesenchymal cell, CTC—chorion trophoblast cell, DEC—decidua cells, HUVEC—human umbilical vascular endothelial cells, CTB—cytotrophoblasts, STB—syncytiotrophoblasts, MYO—myometrium, ENDO—endocervix, TZ—transformation zone, ECTO—ectocervix, MES—cervix mesenchymal cells, VEC—vaginal epithelial cells.

The layout and dimensions of each culture chamber will follow the volume/surface area ratios of the different organ systems to mimic the in vivo environment as closely as possible. The basic design of the microfluidic microchannel array and pillar array to allow localized cell loading and culture while allowing active cell migration, localized stimulant/drug treatment, and biochemical analyses. Each culture chamber will have inlets/outlets to load cells, apply culture media and stimulants, take out effluent for assays, and to conduct end-point immunofluorescent staining of the cells in the chip. To simplify the device operation without the need for tubing or syringe pumps (except for the decidua chamber), a media/effluent reservoir array block can be placed on top of the cell culture chamber layer so that most operations can be conducted through pipet-based cell/reagent handling. This mode of simple pumpless operation allows 10-20 devices to be tested in parallel in one experimental run. To incorporate the vaginal microbiome component, a top-bottom co-culture design is used, where a porous membrane filter (pore size: 0.25-0.8 μm) can be placed on top of the vaginal microbiome compartment. This design was selected to create a complete separation from the mammalian cell compartment with only the supernatant to be diffused through, which is easier to achieve using a membrane filter. Initially, vaginal microbiome metabolites can be loaded (to the top reservoir of the vaginal microbiome chamber. Later, vaginal microbial culture can be conducted in the microbiome chamber.

Myometrium chamber and contractility measurement: The onset of contraction (labor) is a key indicator of both normal term and preterm parturition⁹³⁻⁹⁸. As measuring cellular contractility through microscopy is challenging, MYO can be cultured in a flexible microstructure (or embedded within the structure), whereupon contraction, the shape of the microstructures will change. Monitoring the change in the shape of these microstructures can be conducted either through microscopy or through electrical impedance-based detection (for continuous monitoring). Arrays of these microstructures can be placed at the bottom of the myometrium culture chamber. Microstructures, like micropillar array or cell-embedded hydrogel micropatterns, will also be utilized.^{99,100} Integration of trans epithelial electrical resistance (TEER) sensors for permeability assessment: Since many cell layers functions as perfusion barriers, it is possible to integrate a TEER electrode array into each culture compartment so that permeability between the neighboring compartments can be monitored electronically and continuously (vs. fluorescent dye-based periodic monitoring of permeability).

Gravitas Pregnancy Chip device assembly and operation: The device can be coated with Entactin-collagen Type IV-laminin at a concentration of 10 μg/mL, making the microfluidic channels function as basement membrane barriers as in utero, or collagen Type I to model the placenta stroma. Primary collagen isolated from the amnion portion of term, not in labor, fetal membrane is added to AMC and CTC cell cultures to model the in utero environment. Cells can then be loaded into the OOC device. The reservoir block on top of the OOC device (see FIGS. 2D and 3B) is accessible through regular pipette operation for culture media perfusion and effluent collection. The OOC model can be microfabricated in a combination of polydimethylsiloxane (PDMS), polystyrene (PS), and glass. The dimension of the OOC model can be 4×6 (or scaled smaller or larger), so four chips will fit within a 6-well culture plate format. Since the OOC device is fully compatible with microscopy, time-lapse imaging and/or end-point imaging can be utilized for monitoring the cultured cells within the device.

FIGS. 3A to 3E show Gravitas Pregnancy Chip characterization of cell-cell interactions and cell-specific markers. FIG. 3A: Brightfield images of cells within a Gravitas Pregnancy Chip after 72 hours. FIG. 3B: From left to right: Brightfield images showing EVT invasion into the pillar array separating the placenta trophoblasts from the maternal decidua. Calcein AM (live-green) and Ethidium bromide (dead-red) staining showing migrated cells inside pillar arrays are viable. This is a chamber that can be incorporated into this device to model the first and second trimester of pregnancy. FIG. 3C: Co-migration of Endo (cytokeratin [CK]-18) and Ectocervical (CK-14) cells into the pillar array forming the transformation zone (TZ) of the cervix. The TZ co-expresses both CK-14/CK-18. FIG. 4D: Microchannels separating the decidua (DEC) and myometrium (MYO) chambers show tight cell-cell interfaces. FIG. 3E: Fluorescent images of cells isolated from vaginal-cervix, fetal membrane, placenta, or myometrium tissue Immunostained for cell-specific markers within the POC. AEC—Vimentin (Vim) (green) CK-18 (red), AMC—Vim (green), CTC—HLA-G (red), DEC—Vim (green), HUVEC—Mucin-18 (green), CTB—CK-7 (red), STB—less expression of CK-7 than CYTO (red), MYO—α-smooth muscle actin (α-SMA) (red), MES—Vim (green), ECTO—CK-14 (green), ENDO—CK-18 (red), VEC—Pan-CK (red).

Microfluidic testing of the OOC device: The microfluidic functionality of the OOC can be tested first by conducting permeability assays between neighboring cell culture compartments using fluorescent tracer molecules (e.g., FITC-dextran)¹⁰¹. This allows for the assessment of how fast molecules diffuse between compartments. Based on this permeability profile, the number of interconnecting microfluidic channels and/or dimensions can be optimized to control diffusion. Next, the functionality of the maternal decidua compartment can be tested, the only compartment that is connected to a syringe pump. Decidual supply of nutrients and other essentials, as well as propagation of immune cells and inflammatory mediators at the placental and fetal membrane interfaces, can be used. A fluorescent dye can be used to determine the kinetics of stimulant flow into and out of the decidual chamber.

Cell source, loading, culture, and analyses in the device: Cells described here in above can be used here. Cell concentration in each chamber will resemble the different tissue layer thicknesses seen in utero. Cell loading efficiency and uniformity of cells can be defined within each culture compartment. Next, their viabilities can be assessed over a variety of timepoints (e.g., 6, 12, 24, 36, 48, and 72 hours) to determine how long the cell culture can continue without media replenishment. Since supernatant extraction from each cell culture compartment is needed for various biomarker analyses, a test pipette operation can be used to extract supernatant from each compartment and assess the volume that can be extracted from each compartment. Device dimensions can be optimized as needed.

Gravitas Pregnancy Chip experimental validation. Gravitas Pregnancy Chip devices are cultured under 4 conditions: 1) containing all base cell types, 2) all base cell types with the addition of immune cells in the Fetal, MYO, DEC, and MES chambers, 3) base cells+immune cells+vaginal microbiome conditioned media in the VEC chamber, and 4) base cells+vaginal microbiome conditioned media in the VEC chamber. These 4 conditions will be compared to standard 2D culture, non-human primate tissue (NHP), and human databases to determine which condition is most physiological. Molecular and histochemical-based endpoint assays for these 4 conditions surround the diagram.

For example, to validate OOC functions and develop a 3rd trimester pregnancy model. Pregnancy cannot be fully studied using a single organ system¹⁰². Animal models do not always mimic human pregnancy and particularly parturition^{103,104}, and PTB induced in many of these models are not a naturally occurring pathobiological process¹⁰⁵. To overcome this, OOC technology has been used to recreate the placenta^{75, 77, 78, 80, 106, 107}, fetal membrane¹⁰⁸⁻¹¹⁴, cervix^{86, 115}, vagina^{116, 117}, and adult brain models^{101, 118-120}, but no OOC model that interconnects some or all of these organ systems exist, limiting their usefulness. Cells are used and integrated into the OOC platform to establish an early 3rd trimester pregnancy model. The maternal decidua in this model can form the junction for interconnecting the uterine tissues^{27, 121, 122}, where various factors will propagate from the mother to all in utero compartments (i.e., cervix, myometrium, placenta, fetal membrane). Blood vessels within the decidual-fetal interface (placenta and fetal membranes) transport of nutrients, oxygen, and endocrine factors throughout the intrauterine cavity, to the fetus and help maintain pregnancy^{27, 122}. To mimic this dynamic fluidic transport state, the decidua chamber will accommodate the dynamic (constant) flow of nutrient-enriched media, at or about physiological conditions, similar to that reported in other reproductive biology OOC models^{75, 78, 123}. Besides ensuring that the cells are in their endocrine milieu, there are 2 other independent but vital components that provide tolerance at the F-M interfaces, namely the vaginal microbiome and resident immune cells in the intrauterine tissues^{124, 125}. Vaginal microbiome composition, state, and its metabolome constitute the lower genital tract environment¹²⁶⁻¹²⁸. Resident decidual CD45+ immune cells maintain immunologic harmony with fetal tissues¹²⁹⁻¹³¹. These 2 critical components can also be included in the OOC model.

TABLE 2
Third trimester pregnancy characteristics

| | | |
|---|---|---|
| Cell type | Cell specific markers | Endocrine markers |

Cervical Compartment

| | | |
|---|---|---|
| VEC | Pan CK | N/A |
| Ecto | CK-14 | Progesterone, PRA/PRB, PGRMC |
| TZ | CK-14/CK-18 | N/A |
| Endo | CK-18/Muc5a | Progesterone, PRA/PRB, PGRMC |
| Stroma | Vimentin/Col-1 | LH receptors |

Fetal Membrane Compartment

| | | |
|---|---|---|
| AEC | CK-18, E- cadherin | Relaxin, PGRMC1 and 2 |
| MC | Vimentin, N-cadherin | PGRMC2 |
| CTC | CK-18, E- cadherin | Progesterone, Relaxin, PGRMCs |
| DEC | Vimentin, a-smooth muscle actin, | Progesterone-Associated Endometrial |
| | CD34 | Protein, VEGF, prolactin |

Placenta Compartment

| | | |
|---|---|---|
| HUVEC | PECAM-1, vWb factor | Corticotropin-releasing factor, |
| | | Endothelin receptors (ETA, ETB) |
| CTB | CK-18, E-cadherin | hCG, progesterone, estrogen, GnRH, |
| | | b-endorphin, Somatostatin |
| STB | Syndecan 1, hPL, inhibin | human placental lactogen, hCG, |
| | | placental Growth Hormone (GH), |
| | | Aromatase |
| EVT | Syndecan 1, HLA-G, leukocyte- | Thyrotropin Releasing Hormone |
| | associated immuno- globulin-like | |
| | receptor | |

Myometrium Compartment

| | | |
|---|---|---|
| MYO | α-smooth muscle actin, Cx43 | Prolactin |

Fetal brain compartment

| | | |
|---|---|---|
| SVGp12 + HMC3 | glial fibrillary acidic protein | None |
| | (GFAP), IBA1 | |

The following techniques can be used. Immunocytochemistry: enzyme-linked immunosorbent assay, Western blot.
* Additional markers are available for each cell types and can be Included. Many endocrine markers are not listed in the table due to space limitations.

FIGS. 4A to 4E: Gravitas Pregnancy Chip characterization of cell-cell interactions and cell-specific markers. FIG. 4A: Brightfield images of cells within a POC after 72 hours. FIG. 4B: From left to right: Brightfield images showing EVT invasion into the pillar array separating the placenta trophoblasts from the maternal decidua. Calcein AM (live-green) and Ethidium bromide (dead-red) staining showing migrated cells inside pillar arrays are viable. FIG. 4C: Co-migration of Endo (cytokeratin [CK]-18) and Ectocervical (CK-14) cells into the pillar array forming the transformation zone (TZ) of the cervix. The TZ co-expresses both CK-14/CK-18. FIG. 4D: Microchannels separating the decidua (DEC) and myometrium (MYO) chambers show tight cell-cell interfaces. FIG. 4E: Fluorescent images of cells isolated from vaginal-cervix, fetal membrane, placenta, or myometrium tissue Immunostained for cell-specific markers within the POC. AEC—Vimentin (Vim) (green) CK-18 (red), AMC—Vim (green), CTC—HLA-G (red), DEC—Vim (green), HUVEC—Mucin-18 (green), CTB—CK-7 (red), STB—less expression of CK-7 than CYTO (red), MYO—α-smooth muscle actin (α-SMA) (red), MES—Vim (green), ECTO—CK-14 (green), ENDO—CK-18 (red), VEC—Pan-CK (red).

Model testing and validation. OOC preparation, cell seeding, and cell culture conditions and cellular characteristics: Before seeding cells into the chip, respective microchannels can be filled with Type 1 or 4 collagen to mimic different extra-cellular matrices of the placenta, fetal membranes, and cervix. Since most of the cells in utero reside in a 3D format, cells can be mixed with collagen and then seeded into the OOC device. Initial seeding densities can vary based on time point and endpoint assay. The migration of cells through the microchannels can be monitored using time-lapse live microscopy to assess cellular transitions and the formation of cell-cell barriers.

Cell viability: Lactate dehydrogenase levels in the culture supernatant will determine viability where, generally, a viability value of 80% or less is unacceptable, and experiments can be repeated.

Establish normal cell state of pregnancy physiology: After the systems achieve confluency (70%-80%) with good viability (>90% cells), cellular characteristics and physiological endocrine milieu expected in the 3rd trimester of pregnancy can be assessed (Table 2). To mimic the endocrinologic conditions in the gestational tissues, the placental, myometrial, and cervical culture chambers can be exposed to 3rd trimester levels of progesterone (200 to 400, e.g., 300 ng/mL) and estradiol (2,000 to 4,000, e.g., 6000 μg/mL)^{132,133}.

Cellular transitions: Cellular transition involves cell migration and often contributes to forming barriers (placenta) and specific zones (e.g., transformation zone in the cervix). AEC, CTC, and CTB are expected to show migratory patterns within the OOC and exhibit cellular transitions (epithelial to mesenchymal (EMT) that aid their migration. EMT is a frequently reported cellular-level change that can be determined by cell shape index morphometry, cytokeratin-18, E-cadherin (epithelial markers), vimentin, and N-cadherin (mesenchymal markers) immunostaining^{134,135}. Cells in OOC are expected to maintain their respective characteristics, but migratory cells in microchannels are expected to demonstrate mesenchymal morphology and markers. These characteristics can be considered as the normal state.

Vaginal microbiome: Vaginal swab samples can be collected from consenting subjects. 10 healthy pregnant women (between 18-35 years of age) are swabbed, with 3 swabs per patient for a total of 30 swabs can be taken. A sterile speculum can be placed in the vagina, and vaginal secretions can be collected using sterile swabs. Vaginal swabs can be immediately placed in a tube containing 1 mL pre-reduced anaerobic transport medium, and bacteria can be dissociated from the swabs by vortexing for 5 minutes. Bacterial stocks can be made with 10% glycerol for storage at −80° C. until cultured as described below.

Anaerobic culture of vaginal microbiome/metabolome: A vaginal microbiome can be cultured in vitro to generate a community for use in the OOC. The vaginal microbiome primarily consists of Lactobacilli which are oxygen-tolerant anaerobes and other strict anaerobes^{136,137}. Therefore, strict anaerobic conditions are maintained for in vitro culturing of vaginal microbiome samples in batch reactors¹³⁸. Cell suspensions can be used for metagenome and metabolite analysis. 16S rRNA sequencing can be used to analyze the composition of the in vitro cultured vaginal microbial community (VMC) and compare it to the in vivo VMC (i.e., from DNA directly isolated from fresh vaginal swabs before culture). The VMC composition and relative abundance before and after in vitro culture can be compared to assess the extent of similarity between the cultured community and the freshly isolated community. In parallel, the metabolomic function of the cultured community can be analyzed as the in vivo VMC. A High-resolution LC-MS/MS approach can be used to identify the metabolites present in the samples¹³⁸⁻¹⁴¹. The above culture-sequence-metabolome analysis cycle can be iteratively carried out to identify optimal culture conditions that help generate an in vitro community that mimics the in vivo community. As a secondary validation, culture samples from the optimal culture conditions and the in vivo samples will also be used for shotgun sequence analysis (“deep sequencing”) to obtain a more in-depth coverage of the microbiome. These VMC preparations can be used in OOCs as described herein.

Addition of immune cell components into the OOC: Another critical component that maintains tolerance at the 2 F-M interfaces (placental/decidual and fetal membrane/decidual) is immune cells. Balanced immune interactions by F-M units ensure pregnancy maintenance and feto-placental growth¹⁴²⁻¹⁴⁴. Pregnancy success is determined by regulatory mechanisms at the F-M tissues, ensuring that both the innate and adaptive immune cells aptly support feto-placental growth by controlling inflammation while remodeling uterine tissues¹⁴⁵⁻¹⁵⁴. Conversely, parturition in both humans and animals is associated with physiologic inflammation¹⁵⁵⁻¹⁵⁹ characterized by infiltration and activation of immune cells into the F-M tissues^{144, 160-169}. However, the inclusion of immune cells in any F-M interface in OOC formats has not been previously reported despite its significance.

Inclusion of decidual immune cells and Hofbauer cells: A method reported and used by many labs can be used to obtain decidual cells enriched in CD45+ cells since this approach yields ˜70% decidual cells and 30% CD45+ cells^{170,171}. Decidual CD45+ cells can be isolated from the placenta prior to each culture for inclusion. The villous side of the placenta and cervical stroma chambers in the OOC model can contain PMA-treated THP-1 cell lines. These cells can be included as a substitute for placental Hofbauer and cervical macrophages, as they have already been tested for their functional properties in both organ systems by the present inventors. Protocols to isolate placental and cervical macrophages exist¹⁷², and can be used to test them in the system of the present invention. An alternate approach is to use microglial cells to be included in the fetal brain chamber, as fetal brain inflammation has been reported as one of the major complications of infection-induced preterm birth¹⁷³⁻¹⁷⁷.

Cell physiology and characteristic changes: The presence of microbial metabolites in the vaginal compartment and immune cells in decidual/cervical/placental compartments represents 2 key components of the model system.

Demonstration of normal pregnancy phenotype: Cell/molecular biology experiments and biomarker screening can be done to ensure that expected phenotype and endocrine characteristics are maintained (see Table 2). Immune cell migration and phenotyping by Cytometry by Time Of Flight (CyTOF) analysis: Preliminary CD45+ cell trafficking studies between the maternal/fetal compartments in the OOC device in response to an infectious stimulus (lipopolysaccharide [LPS]), to mimic bacterial infection, was conducted. In this model, LPS increased CD45+ cell influx into the fetal side and increased inflammation. Similar experiments can be repeated, and cell marker specificity can be tested through flow cytometry and immunocytochemistry¹⁷⁸. To study immunophenotypic changes associated with migrating immune cells, a multiplex tissue imaging technology can be used, e.g., Imaging Mass Cytometry (IMC) at an unprecedented resolution¹⁷⁹. IMC allows highly multiplexed, metal-based detection of 30 different protein markers at the same time, and enables high-dimensional, single-cell analysis of complex cell types without any background noise¹⁸⁰⁻¹⁸⁴ (see Table 3 for a list of key/exemplary antibodies). For IMC, the top PDMS part can be removed so that fixed/attached cells stay on the histological glass slides on which the OOC device is built. IMC technology can be introduced to the OOC field, which allows for the analysis of cellular-level changes in OOC.

TABLE 3
Immune cells panel for CyTOF

| Leukocytes | CD45⁺ | |
|---|---|---|
| M2 macrophages | CD45⁺, CD206/CD209 | |
| M1 macrophages | CD45⁺, CD86/CD80 | |
| NK cells | CD45⁺, CD56/CD16⁺/CD3⁻ | |
| Neutrophils | CD45⁺, CD15/CD66B/CD14⁻ | |
| B lymphocytes | CD45⁺, CD19/CD20 | |
| T lymphocytes | Treg | CD45⁺,CD3/CD4/ CD25 |
| | TH | CD45⁺, CD3/CD4 |

| | |
|---|---|
| TH1 | INF-1 |
| TH2 | IL-4 |
| TH17 | IL-17 |

| | |
|---|---|
| CTL | CD3/CD8 |

FIGS. 4A to 4C show immune cell characteristics within the Gravitas Pregnancy Chip. FIG. 4A: Brightfield images of cells chambers containing co-cultured immune cells within an OOC after 72 hours. FIG. 4B: Fluorescent stitched images of the MES, ENDO, DEC, STB, and CTC chambers stained with DAPI (blue), Vimentin (green), and CD45 (red) to localize immune cells. Red CD45-stained immune cells predominantly stayed in resident DEC, MES, MYO, and Fetal chambers, while some cells crossed into neighboring chambers (yellow arrows or boxes). FIG. 4C: HL-60 neutrophils and THP1 macrophages were identified based on live cell videos by size, morphology, and movement.

Exemplary collection protocols for various sample assays/analyses are found in Table 4. Defining normal pregnancy within the Gravitas Pregnancy Chip platform. The following analyses (endocrinology, transcriptome, and proteome) will define normal pregnancy characteristics in the integrated OOC platform. Endocrinology of pregnancy: F-M interfaces are the major sites for both steroid and protein hormone production, and these mediators control F-M physiology during pregnancy, help feto-placental growth, and establish connections between fetal neuronal and endocrine factors that are required for normal maintenance of pregnancy^{2,121,185}. This is one of the reasons for utilizing fetal neuronal cells in this study as the main fetal cell component. Endocrine mediators are distinct in each organ system and can differ even between cells within the system. When the entire 3rd trimester pregnancy cell system is established in the OOC, various endocrine mediators as listed in Table 2 to reflect the normal state of pregnancy can be determined.

TABLE 4
Approaches to collect samples for analysis

| Protocols | Cells | Media |
|---|---|---|
| Immunostaining | Fixed on OOC | N/A |
| | Stained Imaged | |
| Western blot | Accutase Run on Jess | N/A |
| Flow cytometry | Accutase | N/A |
| | Run on CytoFlex | |
| RNAseq | Accutase | N/A |
| | RNAlater Flash frozen | |
| | Store −80° C. | |
| Proteomics | Accutase | Methanol purification |
| | Methanol purification | Store −20° C. |
| | Store −20° C. | |
| CyToF | Fixed on OOC | N/A |
| | Stained Imaged | |
| Immune cell | Fixed on OOC | N/A |
| trafficking | Stained Imaged | |
| Multiplex- | N/A | Flash frozen |
| Cytokines | | Store −80° C. |
| | | Luminex |
| Endocrine | N/A | Flash frozen |
| factors | | Store −80° C. |
| | | Luminex |
| MMP activation | Fixed on OOC | Flash frozen |
| | Stained Imaged | Store −20° C. |
| | | Zymography |

Profiling of normal transcriptome and proteome for each system: Cells can be OOC are established with their morphologic, immunologic, and endocrinologic characteristics that are associated with normal pregnancy. This system can include both vaginal microbiome and immune components. After developing this unique system, a system-specific pregnancy atlas comprising transcriptome and proteome profiles of each organ system is generated for the OOC environment. The multiple cell lines can be collected and pooled into five compartments representing the 1) placenta (STB-CTB-HUVEC), 2) fetal membrane (AEC, AMC, CTC), 3) decidua, 4) myometrium, 5) cervix (ECTO, TZ, ENDO), 6) vagina, 7) AF, and 8) fetal brain. This profile can be used to compare data from parturition and PTB. Although tissue-specific transcriptome and proteome profiles exist, limitations of those data (single tissue analysis, postpartum samples, and loss of inter-tissue interactions) do not allow transcript and protein mapping either within or across different uterine tissues. The newly generated OMICs atlas can be used to define the normal physiologic state of organ systems in the OOC. FIGS. 6A and 6B show proteomics and transcriptomics comparison of the pregnancy chip with non-human primate models.

Next-generation sequencing: For transcriptomics, total RNA can be extracted, quantitated, and integrity validated. High-throughput RNA sequencing can be performed (Illumina NextSeq550) with the depth of sequencing at least 20 million 150-base-long reads/sample. The proteomics analysis workflow: To generate the ion library used in the Sequential Window Acquisition of All Theoretical (SWATH) mass spectra analysis, cell protein lysates (from OOC) can be reduced, alkylated, and trypsinized. For SWATH acquisition, the 5600 Triple TOF can be operated in a looped product ion mode. Using an isolation width of 26 m/z, a set of 32 overlapping windows (1 m/z overlap) can be constructed covering the mass range of 400 to 1200 m/z. To generate the ion libraries, the mass spectra can be processed using the ProteinPilot software (Sciex, Framingham, MA) and the paragon algorithm searched against a human SwissProt database. For SWATH processing, SWATH Acquisition Microapp (version 2.0) within PeakView (RRID: SCR_015786; version 2.2) can be used.

FIGS. 5A to 5C show on-chip microbiome chamber and contractile pillars. FIG. 5A shows a schematic of vaginal epithelial cell (VEC) and microbiome chamber separated by a semipermeable membrane. FIG. 5B shows an illustration of the flexible pad on which myometrium cells sit for easy contractility measurement. FIG. 5C shows a 3D-printed PDMS microstructure. FIG. 5D shows myometrial (MYO) cells being cultured on top of the microstructure.

Quantitative analysis of transcriptomes and proteomes: Quantitative analyses of proteomic and transcriptomic data can be performed at both protein and gene levels. The CLC Genomics Workbench v. 20 can be used for bioinformatic analysis of RNA-Seq data. Filtered sequencing reads will then be processed using the “RNA-Seq Analysis” module by the local alignment against the Homo sapiens (hg38) reference genome with annotated genes and transcripts at minimum matching length and similarity fraction at 90%. Resulting gene counts can be normalized based on fragments/kb of transcript/million mapped reads, and differential expression analysis on the complete list of transcripts can be used to evaluate the fold change between the different sample groups as well as false discovery rate P values. Protein expression can be calculated based on the intensity values using the “proteomic ruler” approach¹⁸⁶. All significantly altered genes and selected gene clusters will undergo pathway analysis using the Ingenuity Pathway Analysis¹⁸⁸ to identify over-represented pathways and to characterize the effects of the changes observed in the gene/protein expression profiles. The resulting associations can be visualized in a network using Cytoscape 3.8.0¹⁸⁹.

FIG. 6A shows differential proteomic analysis of non-human primate tissue compared to pregnancy chip derived cells after 72 hours in culture. The Log-fold changes and adjusted p-values for differential analysis of mass-spectrometry based proteomics profiles across pregnancy chip and NHP was calculated using moderated t-tests provided in the R package limma. Similar methods were applied for RNA-seq transcriptomics comparisons. Percentage of similarity between human pregnancy and pregnancy chip were recorded at each of the cellular, tissue and system levels at transcriptomic, metabolomic and proteomic domains. Interactions corresponding to inter-cellular, inter-tissue and inter-system (fetal vs. maternal) levels at each omics domain will be studied individually for POC, human and NHP tissues using clustering with principal component analysis as well as network visualization methods. This statical analysis was used to create a graphic image (shown FIG. 6B) when the pregnancy chip was over 80% similar to nonhuman primate tissue in all of the organs.

A coordinated sequence of events can be determined for each chamber representing F-M tissues that resemble normal, healthy 3^{rd} trimester pregnancy. Physiologic alterations occurring at various cell levels are communicated between the F-M systems, and each cell-level change that maintains uterine homeostasis during human pregnancy can be determined. Endocrine, immune, and other cellular metabolic changes in different systems are not restricted to that unit alone; thus, the OOC model will portray the best physiologic state of the entire uterine system when all the systems are in place.

Parturition OOC model. Parturition is a physiologic alteration of various systems' homeostatic balances. The present invention can be used to address the initiator of events that disturb this balance to promote a parturition phenotype in each system. The key question still debated in the field is what the initiator of parturition is: 1) fetal signaling^{6,209,210}, 2) maternal signaling^{27,121}, or 3) synchronized F-M signaling^{4,93,211}. As F-M units have been established by the present inventors, the integrated OOC can address this long-standing question.

Potential initiators. 1) Fetal organ maturation can release biochemicals into the AF that include, but are not limited to, surfactants from the lung^{6,212-214}, endothelins from the kidney^{215,216}, brain-derived nerve factor from brain cells²¹⁷, and platelet activation factor from the liver²¹⁸. These molecules are proinflammatory and cause an increase in proinflammatory cytokines in the AF; specifically, tumor necrosis factor (TNF)α and interleukin (IL)-1β are capable of inducing parturition-associated changes in uterine tissues due to their ability to increase pro-parturition-associated signaling in both F-M compartments and increase oxidative stress (OS and increase reactive oxygen species) in fetal membranes and placenta²¹⁹⁻²²¹. These inflammatory molecules in AF and reactive oxygen radicals induce fetal membrane damage, which can set off a cascade of inflammation in other uterine compartments. Specifically, at the placental/decidual and membrane/decidual interfaces where decidua and resident immune cells function as amplifiers of fetal biochemical, inflammatory and reactive oxygen species signals increase the inflammatory load, creating immune intolerance on the maternal side^{5, 104, 121, 212, 220}. 2) Maternal biologic mechanisms responding to endocrine and inflammatory changes can initiate inflammatory changes in decidua and propagate them towards fetal compartments, increasing fetal inflammation. 3) Both F-M signals originate simultaneously and contribute to parturition. Importantly, regardless of the signal's origin, they all propagate towards the decidua, the junction where inflammation is amplified. Decidual activation causing immune cells and other proinflammatory mediators to propagate in both directions tilts the threshold of immune balance.

TABLE 5
Determining parturition phenotype

| | |
|---|---|
| Cervix | Collagen degradation |
| | MMP9 and MMP7 assay |
| | Prostaglandin E2 and F2a |
| | Inflammation Pro/anti- inflammatory cytokine |
| Fetal Membrane | Cell fate (necrosis, apoptosis and senescence) |
| | Collagen degradation MMP9 assay |
| | CD45+ cell infiltration into chorion |
| | Inflammation Pro/anti- inflammatory cytokine |
| | Prostaglandin production |
| Decidua | Inflammation Pro/anti- inflammatory cytokine |
| | CD45+ activation |
| Placenta | corticotropin-releasing hormone |
| | Decidual NK cell migration and activation |
| | Cell fate (necrosis, apoptosis and senescence) |
| | Inflammation Pro/anti- inflammatory cytokine |
| Myometrium | Contractility markers Conexin-43, COX2, OXTR |
| | PRA/PRB Switch |
| | Inflammation Pro/anti- inflammatory cytokine PGE2 and PGF2a |
| Fetal brain | Normal phenotype Neuron and glial markers |
| Amniotic fluid | Inflammation Pro/anti- inflammatory cytokine |
| | CD45+ cells increase |

Assays for phenotype determination: ELISA, immunocytochemistry, Western blot, CyTOF analysis, receptor and signaling marker analysis, reporter cell assays

The OOC device can be prepared with AF, vaginal microbiome, and immune components. 1) Fetus triggering parturition: To create a parturition phenotype using fetal signals of organ maturation, fetal membrane and placental cells in the OOC device can be exposed to surfactant (6 ng/mL), and endothelin (40-80 pmol/L), and platelet-activating factor (5 ng/mL), which are fetal signals of organ maturation^{215, 222, 223}. To mimic fetal signals of organ maturation-induced inflammation, AF can be reconstituted with 50 ng/mL of TNFα and 50 ng/mL IL-1β. These concentrations were selected based on the reported concentrations of these cytokines in the AF at term labor²²⁴⁻²²⁷. Although IL-6 and IL-8 increases have been reported, their mechanistic contributions to labor are debated. Besides inflammatory mediators in AF, feto-placental compartments are expected to experience increased OS. This condition can be recreated by treating AEC and CTB with H₂O₂ (100 μM) or TNFα (50 ng/ml) based on reported values²²⁸. Both mediators are expected to cause OS-induced damage resulting in inflammation. Fetal membranes and placenta experience OS at term. Although HUVEC cells are also shown to have OS, this is reported as secondary phenomenon to primary events in the AF and fetal tissue compartments. Therefore, natural progression of OS-induced changes in the HUVEC/placental compartments is monitored with the OOC device.

2) Mother triggering parturition: In the 2^{nd} scenario, maternal biologic effects triggering parturition are determined, primarily initiated in myometrial and cervical tissues. To promote a parturition phenotype, both cervical and myometrial cultures in OOC can be treated with 30 μg/mL of oxytocin and 300 μg/mL of prostaglandin F2α. These concentrations are based on the levels found in maternal plasma during term labor²²⁹. Oxytocin and prostaglandin stimulate uterine contractions, cervical ripening, and dilatation during pregnancy²³⁰⁻²³¹. 3) Feto-maternal units synchronously triggering parturition: The third scenario tests F-M compartments triggering parturition at the same time. Treatments #1 and #2 described above can be applied together to determine the combined effect. In all three scenarios, cultures can be set up as detailed hereinabove (e.g., 24 hours for cell attachment, 48 hours to establish baseline, 72 hours total), and development of parturition-associated phenotype can be assessed for an additional 72 hours through supernatant and cell analyses. Parturition-associated markers can be screened in each system as described in Table 5.

AF metabolomic analyses can be performed to get a deeper phenotype of parturition with each cell type and for each system. Myometrial measurements as described above can be performed to confirm contractility.

Human parturition results from distinct responses from different compartments that are well coordinated and synchronized to empty the uterus. The generalizable aspect of response in each tissue system is reflected by inflammation; however, inflammation is a heterogenous state in each tissue indicative of a transition from a quiescent state to an active state. As shown in Table 3, inflammatory profiles examined will indicate system-specific activation, and changes in every organ system measured. A 3-fold change in various marker expressions, along with contractility changes in myometrium, can be interpreted as a proinflammatory shift. Besides, direct measurements of TEER and electric impedance, which can assess the barriers/permeability in specific tissue compartments, can be used to monitor parturition preparedness by those tissues

TABLE 6
Determining preterm birth phenotype characteristics

| Cell type | Characteristics |
|---|---|
| Cervix | M2- to M1 switch for macrophages |
| | Prostaglandin E2 and F2a |
| | Inflammation Pro/anti- inflammatory cytokine MMP9/MMP7 and |
| | TLR4, NF-kB activation |
| Fetal Membrane | Collagen degradation |
| | MMP9 stain |
| | Cytologic chorioamnionitis |
| | Inflammation Pro/anti- inflammatory cytokine MMP9/MMP7 and |
| | TLR4, NF-kB activation |
| | Prostaglandin production |
| Decidua | Inflammation Pro/anti- inflammatory cytokine, TLR4, NF-kB |
| | activation |
| | CD45+ cell infiltration into chorion |
| | CD45+ activation |
| Placenta | Decidual NK cell migration and activation |
| | Inflammation Pro/anti- inflammatory cytokine, TLR4, NF-kB |
| | activation |
| Myometrium | Contractility markers Coonexin-43, COX2, OXTR |
| | PRA/PRB Switch |
| | Inflammation Pro/anti- inflammatory cytokine release PGE2 and |
| | PGF2a |
| | ELISA. ICC for TLR4, |
| | NF-kB activation |
| Fetal brain | Inflammation Pro/anti- inflammatory cytokine release |
| Amniotic fluid | Inflammation Pro/anti- inflammatory cytokine release Immune |
| | cells counts |

Assay approaches will include multiplex assays, immunostaining, flow cytometry, ELISA, reporter cell assays.

Develop an infection-associated preterm birth (PTh) model. PTB is a complex syndrome. The focus is infection-associated spontaneous PTB, which contributes to ˜0.50% of all PTBs. Only a single report has shown an ascending model of infection and PTB recreated using OOC technology⁸¹. This model used lipopolysaccharide (LPS) to simulate infection; however, it was still limited to evaluation of a single organ system (fetal membrane) and ignored, for example, the potential antimicrobial protection offered by maternal cervix and placental barrier functions seen in vivo prior to microbes reaching the membranes. Besides, immune cells in the decidua play a major role in determining inflammatory amplification during microbial invasion. Therefore, use of the proposed integrated OOC platform where LPS can be introduced through the vaginal compartment would be significantly more realistic as seen in vivo and will provide a belier assessment on how infection causes changes in the various F-M. Various microbiological agents can also be tested to simulate bacterial vaginosis, vaginitis and other microbial infection besides LPS.

Pathologic changes associated with infection: Each sub-system can be tested for pathologic changes, particularly those related to inflammation. Specific molecular-level changes to be examined in each compartment are listed in Table 6. Phenotypic overlap between term parturition and LPS-induced PTB phenotype is expected, but the system can also be used to detect distinct molecular-level changes for each cell type and organ system. These differences can lead to understanding the mechanisms of parturition at normal term vs preterm.

Immune cell trafficking and phenotyping: Ascending LPS is expected to activate immune cells in decidua, and thus trafficking of activated immune cells is expected to increase towards the fetal compartments. The destination and immunophenotype of the immune cells can be determined by IMC. In addition, a shift (M2 to M1) in the cervical and placenta macrophages, indicating inflammation, can also be detected.

Transcriptomic and proteomic analyses: A comprehensive evaluation of each system after treatment with LPS can be performed.

Reversing the disease model to restore a normal pregnancy phenotype. One way to provide rigor in this experimental model is by reversing the PTB to a normal pregnancy phenotype or like that of untreated controls. A therapeutic dose of 17-hydroxyprogesterone (17-OHP) currently used in the clinic can be used. In the infection-induced PTB model, 17-OHP is expected to reduce contractile and ripening phenotypes (inflammation associated) of myometrium and cervix. In addition to 17-OHP other drugs can be tested for treating infection-induced PTB. The OOC device can be exposed to LPS. To mimic systemic 17-OHP treatment, dynamic (constant) flow of nutrient-enriched media with 17-OHP (0.5 μl/h) can be disseminated into the decidua chamber for 72 hours to mimic repeated treatment as done clinically to allow constant propagation of factors from the mother to the fetus. Cells/media obtained after 24 and 48 hours can be tested for reversal of PTB-associated changes. In addition, each system can be analyzed to determine changes as detailed in Table 5 (term parturition). This example is provided to show that the device of the present invention can be used for any number of preclinical drug trials. The use 17-OHP was used solely as an example.

Integrating bioinformatics to determine the difference between healthy vs disease state: The same analytical strategy and methodology defined for the quantitative analysis of transcriptome and proteomes can be applied here to highlight the differences in normal vs. PTB. Differentially regulated genes (LFC>1.5 and FDR P<0.05) affecting pathways of interest will undergo individual qPCR validation for more precise quantification.

The integrated OOC model is designed to mimic human pregnancy physiology and pathobiology in vitro and can be used to generate a knowledge base far beyond what is available from the study of single cells. Cellularity of the fetal compartment will mimic early 3^{rd} trimester, which is a good representation of all trimesters. Adjusting the various culture chambers (e.g., placenta) can be used to better represent cellularity mimicking different trimesters as well as adjusting the changes in O₂ environment as seen in utero (hypoxic in 1st to hyperoxic in 2nd trimester). A dynamic flow of media is used through the decidual chamber, to mimic maternal blood flow of various nutritional and growth factors required for feto-placental cell growth.

A nonhuman primate (NHP) model of pregnancy is most directly relevant due to the many similarities to human pregnancy such as the reproductive anatomy, number of fetuses (singleton), long gestational period (160-170 days), type and structure of placenta (hemochorial), initiation of labor (hormonal control of parturition), sensitivity to pathogens and timeline of fetal brain development^{243,244}. The present invention can be used to examine NHP pregnancy and to compare such data.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown byway of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Romero R, Dey S K, Fisher S J. Preterm labor: one syndrome, many causes. Science. 2014; 345(6198):760-765.
2. Tal R, Taylor H S. Endocrinology of Pregnancy. In: Feingold K R, Anawalt B, Boyce A, et al., eds. Endotext. South Dartmouth (MA)2000.
3. Rokas A, Mesiano S, Tamam O, LaBella A, Zhang G, Muglia L. Developing a theoretical evolutionary framework to solve the mystery of parturition initiation. Elife. 2020; 9.
4. Smith R. Alterations in the hypothalamic pituitary adrenal axis during pregnancy and the placental clock that determines the length of parturition. J Reprod Immunol. 1998; 39(1-2):215-220.
5. Menon R, Bonney E A, Condon J, Mesiano S, Taylor R N. Novel concepts on pregnancy clocks and alarms: redundancy and synergy in human parturition. Hum Reprod Update. 2016; 22(5):535-560.
6. Mendelson C R, Montalbano A P, Gao L. Fetal-to-maternal signaling in the timing of birth. J Steroid Biochem Mol Biol. 2017; 170:19-27.
7. Beck S, Wojdyla D, Say L, et al. The worldwide incidence of preterm birth: a systematic review of maternal mortality and morbidity. Bull World Health Organ. 2010; 88(1):31-38.
8. Shapiro-Mendoza C K, Lackritz E M. Epidemiology of late and moderate preterm birth. Semin Fetal Neonatal Med. 2012; 17(3):120-125.
9. Simmons L E, Rubens C E, Darmstadt G L, Gravett M G. Preventing preterm birth and neonatal mortality: exploring the epidemiology, causes, and interventions. Semin Perinatol. 2010; 34(6):408-415.
10. Blencowe H, Cousens S, Oestergaard M Z, et al. National, regional, and worldwide estimates of preterm birth rates in the year 2010 with time trends since 1990 for selected countries: a systematic analysis and implications. Lancet. 2012; 379(9832):2162-2172.
11. Villar J, Papageorghiou A T, Knight H E, et al. The preterm birth syndrome: a prototype phenotypic classification. Am J Obstet Gynecol. 2012; 206(2):119-123.
12. Muglia L J, Katz M. The enigma of spontaneous preterm birth. N Engl J Med. 2010; 362(6):529-535.
13. Plunkett J, Muglia L J. Genetic contributions to preterm birth: implications from epidemiological and genetic association studies. Ann Med. 2008; 40(3):167-195.
14. Lockwood C J, Kuczynski E. Markers of risk for preterm delivery. Journal of perinatal medicine. 1999; 27(1):5-20.
15. Romero R, Espinoza J, Kusanovic J P, et al. The preterm parturition syndrome. BJOG. 2006; 113 Suppl 3:17-42.
16. American College of O, Gynecologists' Committee on Practice B-O. Prediction and Prevention of Spontaneous Preterm Birth: ACOG Practice Bulletin, Number 234. Obstet Gynecol. 2021; 138(2):e65-e90.
17. Dammann O, Leviton A. Maternal intrauterine infection, cytokines, and brain damage in the preterm newborn. Pediatr Res. 1997; 42(1):1-8.
18. Jacobsson B. Infectious and inflammatory mechanisms in preterm birth and cerebral palsy. Eur J Obstet Gynecol Reprod Biol. 2004; 115(2):159-160.
19. Bersani I, Thomas W, Speer C P. Chorioamnionitis—the good or the evil for neonatal outcome? J Matern Fetal Neonatal Med. 2012; 25 Suppl 1:12-16.
20. Johanzon M, Odesjo H, Jacobsson B, Sandberg K, Wennerholm U B. Extreme preterm birth: onset of delivery and its effect on infant survival and morbidity. Obstet Gynecol. 2008; 111(1):42-50.
21. McCabe E R, Carrino G E, Russell R B, Howse J L. Fighting for the next generation: US Prematurity in 2030. Pediatrics. 2014; 134(6):1193-1199.
22. Challis J R, Lockwood C J, Myatt L, Norman J E, Strauss J F, 3rd, Petraglia F. Inflammation and pregnancy. Reprod Sci. 2009; 16(2):206-215.
23. Armistead B, Oler E, Adams Waldorf K, Rajagopal L. The Double Life of Group B Streptococcus: Asymptomatic Colonizer and Potent Pathogen. J Mol Biol. 2019; 431(16):2914-2931.
24. Elovitz M A, Mrinalini C. Animal models of preterm birth. Trends Endocrinol Metab. 2004; 15(10):479-487.
25. Chauhan S P, Ananth C V. Periviable births: epidemiology and obstetrical antecedents. Semin Perinatol. 2013; 37(6):382-388.
26. Lawn J E, Kinney M V, Belizan J M, et al. Born too soon: accelerating actions for prevention and care of 15 million newborns born too soon. Reprod Health. 2013; 10 Suppl 1:S6.
27. Pavlicev M, Norwitz E R. Human Parturition: Nothing More Than a Delayed Menstruation. Reprod Sci. 2018; 25(2):166-173.
28. Frey H A, Klebanoff M A. The epidemiology, etiology, and costs of preterm birth. Semin Fetal Neonatal Med. 2016; 21(2):68-73.
29. Outcomes IoMUCoUPBaAH. Preterm Birth: Causes, Consequences, and Prevention. Washington DC: National Academies Press; 2007.
30. Myatt L, Eschenbach D A, Lye S J, et al. A standardized template for clinical studies in preterm birth. Reprod Sci. 2012; 19(5):474-482.
31. Park H J, Cho H Y, Cha D H. The Amniotic Fluid Cell-Free Transcriptome Provides Novel Information about Fetal Development and Placental Cellular Dynamics. Int J Mol Sci. 2021; 22(5).
32. Di Renzo G C, Rosati A, Sarti R D, Cruciani L, Cutuli A M. Does fetal sex affect pregnancy outcome? Gend Med. 2007; 4(1):19-30.
33. Challis J, Newnham J, Petraglia F, Yeganegi M, Bocking A. Fetal sex and preterm birth. Placenta. 2013; 34(2):95-99.
34. Myatt L, Maloyan A. Obesity and Placental Function. Semin Reprod Med. 2016; 34(1):42-49.
35. Ingemarsson I. Gender aspects of preterm birth. BJOG. 2003; 110 Suppl 20:34-38.
36. Teoh P J, Ridout A, Seed P, Tribe R M, Shennan A H. Gender and preterm birth: Is male fetal gender a clinically important risk factor for preterm birth in high-risk women? Eur J Obstet Gynecol Reprod Biol. 2018; 225:155-159.
37. McGregor J A, Leff M, Orleans M, Baron A. Fetal gender differences in preterm birth: findings in a North American cohort. Am J Perinatol. 1992; 9(1):43-48.
38. Varner M W, Costantine M M, Jablonski K A, et al. Sex-Specific Genetic Susceptibility to Adverse Neurodevelopmental Outcome in Offspring of Pregnancies at Risk of Early Preterm Delivery. Am J Perinatol. 2020; 37(3):281-290.
39. Spong C Y, lams J, Goldenberg R, Hauck F R, Willinger M. Disparities in perinatal medicine: preterm birth, stillbirth, and infant mortality. Obstet Gynecol. 2011; 117(4):948-955.
40. Menon R, Dunlop A L, Kramer M R, Fortunato S J, Hogue C J. An overview of racial disparities in preterm birth rates: caused by infection or inflammatory response? Acta Obstet Gynecol Scand. 2011; 90(12):1325-1331.
41. Sparks P J. Do biological, sociodemographic, and behavioral characteristics explain racial/ethnic disparities in preterm births? Soc Sci Med. 2009; 68(9):1667-1675.
42. Kramer M S, Wilkins R, Goulet L, et al. Investigating socio-economic disparities in preterm birth: evidence for selective study participation and selection bias. Paediatr Perinat Epidemiol. 2009; 23(4):301-309.
43. Menon R. Spontaneous preterm birth, a clinical dilemma: etiologic, pathophysiologic and genetic heterogeneities and racial disparity. Acta Obstet Gynecol Scand. 2008; 87(6):590-600.
44. Hogue C J, Menon R, Dunlop A L, Kramer M R. Racial disparities in preterm birth rates and short inter-pregnancy interval: an overview. Acta Obstet Gynecol Scand. 2011; 90(12):1317-1324.
45. Romero R, Velez Edwards D R, Kusanovic J P, et al. Identification of fetal and maternal single nucleotide polymorphisms in candidate genes that predispose to spontaneous preterm labor with intact membranes. Am J Obstet Gynecol. 2010; 202(5):431-434.
46. Burris H H, Collins J W, Jr. Race and preterm birth—the case for epigenetic inquiry. Ethn Dis. 2010; 20(3):296-299.
47. Menon R, Pearce B, Velez D R, et al. Racial disparity in pathophysiologic pathways of preterm birth based on genetic variants. Reprod Biol Endocrinol. 2009; 7:62.
48. Menon R, Velez D R, Morgan N, Lombardi S J, Fortunato S J, Williams S M. Genetic regulation of amniotic fluid TNF-alpha and soluble TNF receptor concentrations affected by race and preterm birth. Hum Genet. 2008; 124(3):243-253.
49. Menon R, Camargo M C, Thorsen P, Lombardi S J, Fortunato S J. Amniotic fluid interleukin-6 increase is an indicator of spontaneous preterm birth in white but not black Americans. Am J Obstet Gynecol. 2008; 198(1):77-77.
50. Menon R, Arora C P, Hobel C J, Fortunato S J. Corticotrophin-releasing hormone in lipopolysaccharide-stimulated term fetal membranes and amniotic fluid from term and preterm birth in African Americans and Caucasians. Reprod Sci. 2008; 15(5):477-483.
51. Brown H L, Chireau M V, Jallah Y, Howard D. The “Hispanic paradox”: an investigation of racial disparity in pregnancy outcomes at a tertiary care medical center. Am J Obstet Gynecol. 2007; 197(2):197-197.
52. Simhan H N, Krohn M A, Roberts J M, Zeevi A, Caritis S N. Interleukin-6 promoter-174 polymorphism and spontaneous preterm birth. Am J Obstet Gynecol. 2003; 189(4):915-918.
53. Fiscella K. Racial disparities in preterm births. The role of urogenital infections. Public Health Rep. 1996; 111(2):104-113.
54. Green C A, Johnson J D, Vladutiu C J, Manuck T A. The association between maternal and paternal race and preterm birth. Am J Obstet Gynecol MFM. 2021; 3(4):100353.
55. Tolcher M C, Sangi-Haghpeykar H, Mendez-Figueroa H, Aagaard K M. Low-dose aspirin for preeclampsia prevention: efficacy by ethnicity and race. Am J Obstet Gynecol MFM. 2020; 2(4):100184.
56. Rappoport N, Toung J, Hadley D, et al. A genome-wide association study identifies only two ancestry specific variants associated with spontaneous preterm birth. Scientific reports. 2018; 8(1):226.
57. MacDorman M F. Race and ethnic disparities in fetal mortality, preterm birth, and infant mortality in the United States: an overview. Semin Perinatol. 2011; 35(4):200-208.
58. Jesse D E, Swanson M S, Newton E R, Morrow J. Racial disparities in biopsychosocial factors and spontaneous preterm birth among rural low-income women. J Midwifery Womens Health. 2009; 54(1):35-42.
59. Hamilton B E, Martin J A, Ventura S J. Births: preliminary data for 2005. Natl Vital Stat Rep. 2006; 55(11):1-18.
60. Fisher S J, Cui T Y, Zhang L, et al. Adhesive and degradative properties of human placental cytotrophoblast cells in vitro. J Cell Biol. 1989; 109(2):891-902.
61. Kliman H J, Feinberg R F, Haimowitz J E. Human trophoblast-endometrial interactions in an in vitro suspension culture system. Placenta. 1990; 11(4):349-367.
62. Kliman H J, Nestler J E, Sermasi E, Sanger J M, Strauss J F, 3rd. Purification, characterization, and in vitro differentiation of cytotrophoblasts from human term placentae. Endocrinology. 1986; 118(4):1567-1582.
63. Menon R, Boldogh I, Urrabaz-Garza R, et al. Senescence of primary amniotic cells via oxidative DNA damage. PLoS One. 2013; 8(12):e83416.
64. Lim R, Chan S T, Tan J L, Mockler J C, Murphy S V, Wallace E M. Preterm human amnion epithelial cells have limited reparative potential. Placenta. 2013; 34(6):486-492.
65. Sun K, Ma R, Cui X, et al. Glucocorticoids induce cytosolic phospholipase A2 and prostaglandin H synthase type 2 but not microsomal prostaglandin E synthase (PGES) and cytosolic PGES expression in cultured primary human amnion cells. J Clin Endocrinol Metab. 2003; 88(11):5564-5571.
66. Menon R, Radnaa E, Behnia F, Urrabaz-Garza R. Isolation and characterization human chorion membrane trophoblast and mesenchymal cells. Placenta. 2020; 101:139-146.
67. Lavu N, Richardson L, Radnaa E, et al. Oxidative stress-induced downregulation of glycogen synthase kinase 3 beta in fetal membranes promotes cellular senescencedagger. Biol Reprod. 2019; 101(5):1018-1030.
68. McConkey C A, Delorme-Axford E, Nickerson C A, et al. A three-dimensional culture system recapitulates placental syncytiotrophoblast development and microbial resistance. Sci Adv. 2016; 2(3):e1501462.
69. Kshirsagar S K, Alam S M, Jasti S, et al. Immunomodulatory molecules are released from the first trimester and term placenta via exosomes 1. Placenta. 2012; 33(12):982-990.
70. Taylor R N, Newman E D, Chen S A. Forskolin and methotrexate induce an intermediate trophoblast phenotype in cultured human choriocarcinoma cells. Am J Obstet Gynecol. 1991; 164(1 Pt 1):204-210.
71. Schutte S C, Taylor R N. A tissue-engineered human endometrial stroma that responds to cues for secretory differentiation, decidualization, and menstruation. Fertil Steril. 2012; 97(4):997-1003.
72. El-Farrash R A, Gad G I, Abdelkader H M, Salem D A D, Fahmy S A. Simulated amniotic fluid-like solution given enterally to neonates after obstructive bowel surgeries: A randomized controlled trial. Nutrition. 2019; 66:187-191.
73. Christensen R D, Havranek T, Gerstmann D R, Calhoun D A. Enteral administration of a simulated amniotic fluid to very low birth weight neonates. J Perinatol. 2005; 25(6):380-385.
74. Lima-Rogel V, Ojeda M A, Villegas C, et al. Tolerance of an enterally administered simulated amniotic fluid-like solution by neonates recovering from surgery for congenital bowel abnormalities. J Perinatol. 2004; 24(5):295-298.
75. Yin F, Zhu Y, Zhang M, Yu H, Chen W, Qin J. A 3D human placenta-on-a-chip model to probe nanoparticle exposure at the placental barrier. Toxicol In Vitro. 2019; 54:105-113.
76. Blundell C, Tess E R, Schanzer A S, et al. A microphysiological model of the human placental barrier. Lab Chip. 2016; 16(16):3065-3073.
77. Blundell C, Yi Y S, Ma L, et al. Placental Drug Transport-on-a-Chip: A Microengineered In Vitro Model of Transporter-Mediated Drug Efflux in the Human Placental Barrier. Adv Healthc Mater. 2018; 7(2).
78. Lee J S, Romero R, Han Y M, et al. Placenta-on-a-chip: a novel platform to study the biology of the human placenta. J Matern Fetal Neonatal Med. 2016; 29(7):1046-1054.
79. Miura S, Sato K, Kato-Negishi M, Teshima T, Takeuchi S. Fluid shear triggers microvilli formation via mechanosensitive activation of TRPV6. Nature communications. 2015; 6:8871.
80. Pemathilaka R L, Caplin J D, Aykar S S, Montazami R, Hashemi N N. Placenta-on-a-Chip: In Vitro Study of Caffeine Transport across Placental Barrier Using Liquid Chromatography Mass Spectrometry. Glob Chall. 2019; 3(3):1800112.
81. Richardson L S, Kim S, Han A, Menon R. Modeling ascending infection with a feto-maternal interface organ-on-chip. Lab Chip. 2020.
82. Richardson L, Jeong S, Kim S, Han A, Menon R. Amnion membrane organ-on-chip: an innovative approach to study cellular interactions. The FASEB Journal. 2019; 33(8):8945-8960.
83. Radnaa E, Richardson L S, Sheller-Miller S, et al. Extracellular vesicle mediated feto-maternal HMGB1 signaling induces preterm birth. Lab Chip. 2021; 21(10):1956-1973.
84. Mosavati B, Oleinikov A V, Du E. Development of an Organ-on-a-Chip-Device for Study of Placental Pathologies. Int J Mol Sci. 2020; 21(22).
85. Arumugasaamy N, Rock K D, Kuo C Y, Bale T L, Fisher J P. Microphysiological systems of the placental barrier. Adv Drug Deliv Rev. 2020; 161-162:161-175.
86. Tantengco O A G, Richardson L S, Medina P M B, Han A, Menon R. Organ-on-chip of the cervical epithelial layer: A platform to study normal and pathological cellular remodeling of the cervix. FASEB J. 2021; 35(4):e21463.
87. Wikswo J P, Curtis E L, Eagleton Z E, et al. Scaling and systems biology for integrating multiple organs-on-a-chip. Lab on a chip. 2013; 13(18):3496-3511.
88. Skardal A, Murphy S V, Devarasetty M, et al. Multi-tissue interactions in an integrated three-tissue organ-on-a-chip platform. Scientific reports. 2017; 7(1):8837.
89. McAleer C W, Long C J, Elbrecht D, et al. Multi-organ system for the evaluation of efficacy and off-target toxicity of anticancer therapeutics. Science Translational Medicine. 2019; 11(497):eaav1386.
90. Maoz B M, Herland A, FitzGerald E A, et al. A linked organ-on-chip model of the human neurovascular unit reveals the metabolic coupling of endothelial and neuronal cells. Nature biotechnology. 2018; 36(9):865-874.
91. Edington C D, Chen W L K, Geishecker E, et al. Interconnected Microphysiological Systems for Quantitative Biology and Pharmacology Studies. Scientific reports. 2018; 8(1):4530.
92. Gold K, Gaharwar A K, Jain A. Emerging trends in multiscale modeling of vascular pathophysiology: Organ-on-a-chip and 3D printing. Biomaterials. 2019; 196:2-17.
93. Mesiano S, Chan E C, Fitter J T, Kwek K, Yeo G, Smith R. Progesterone withdrawal and estrogen activation in human parturition are coordinated by progesterone receptor A expression in the myometrium. J Clin Endocrinol Metab. 2002; 87(6):2924-2930.
94. Olson D M, Ammann C. Role of the prostaglandins in labour and prostaglandin receptor inhibitors in the prevention of preterm labour. Front Biosci. 2007; 12:1329-1343.
95. Williams K C, Renthal N E, Gerard R D, Mendelson C R. The microRNA (miR)-199a/214 cluster mediates opposing effects of progesterone and estrogen on uterine contractility during pregnancy and labor. Mol Endocrinol. 2012; 26(11):1857-1867.
96. Sivarajasingam S P, Imami N, Johnson M R. Myometrial cytokines and their role in the onset of labour. J Endocrinol. 2016; 231(3):R101-R119.
97. Brainard A M, Korovkina V P, England S K. Potassium channels and uterine function. Semin Cell Dev Biol. 2007; 18(3):332-339.
98. Li Y, Lorca R A, Ma X, Rhodes A, England S K. BK channels regulate myometrial contraction by modulating nuclear translocation of NF-kappaB. Endocrinology. 2014; 155(8):3112-3122.
99. Ya S, Ding W, Li S, et al. On-Chip Construction of Liver Lobules with Self-Assembled Perfusable Hepatic Sinusoid Networks. ACS Appl Mater Interfaces. 2021; 13(28):32640-32652.
100. Lind J U, Yadid M, Perkins I, et al. Cardiac microphysiological devices with flexible thin-film sensors for higher-throughput drug screening. Lab Chip. 2017; 17(21):3692-3703.
101. Jeong S, Kim S, Buonocore J, et al. A Three-Dimensional Arrayed Microfluidic Blood-Brain Barrier Model With Integrated Electrical Sensor Array. IEEE Trans Biomed Eng. 2018; 65(2):431-439.
102. Smith R. Parturition. N Engl J Med. 2007; 356(3):271-283.
103. Manuel C R, Ashby C R, Reznik S E. Discrepancies in Animal Models of Preterm Birth. Curr Pharm Des. 2017; 23(40):6142-6148.
104. Nielsen B W, Bonney E A, Pearce B D, Donahue L R, Sarkar I N, Preterm Birth International C. A Cross-Species Analysis of Animal Models for the Investigation of Preterm Birth Mechanisms. Reprod Sci. 2016; 23(4):482-491.
105. Nold C, Maubert M, Anton L, Yellon S, Elovitz M A. Prevention of preterm birth by progestational agents: what are the molecular mechanisms? Am J Obstet Gynecol. 2013; 208(3):223 e221-227.
106. Horii M, Touma O, Bui T, Parast M M. Modeling human trophoblast, the placental epithelium at the maternal fetal interface. Reproduction. 2020; 160(1):R1-R11.
107. Pemathilaka R L, Reynolds D E, Hashemi N N. Drug transport across the human placenta: review of placenta-on-a-chip and previous approaches. Interface Focus. 2019; 9(5):20190031.
108. Aronoff D M. DECONSTRUCTING EXTRAPLACENTAL MEMBRANES TO UNDERSTAND BACTERIAL CHORIOAMNIONITIS. Trans Am Clin Climatol Assoc. 2020; 131:72-79.
109. Gnecco J S, Anders A P, Cliffel D, et al. Instrumenting a Fetal Membrane on a Chip as Emerging Technology for Preterm Birth Research. Curr Pharm Des. 2017; 23(40):6115-6124.
110. Richardson L, Kim S, Menon R, Han A. Organ-On-Chip Technology: The Future of Feto-Maternal Interface Research? Front Physiol. 2020; 11:715.
111. Dixon C L, Richardson L, Sheller-Miller S, Saade G, Menon R. A distinct mechanism of senescence activation in amnion epithelial cells by infection, inflammation, and oxidative stress. Am J Reprod Immunol. 2017.
112. Richardson L, Jeong S, Kim S, Han A, Menon R. Amnion membrane organ-on-chip: an innovative approach to study cellular interactions. Faseb j. 2019:fj201900020RR.
113. Richardson L, Gnecco J, Ding T, et al. Fetal Membrane Organ-On-Chip: An Innovative Approach to Study Cellular Interactions. In. Reproductive Sciences 2019.
114. Richardson L S, Kim S, Han A, Menon R. Modeling ascending infection with a feto-maternal interface organ-on-chip. Lab Chip. 2020; 20(23):4486-4501.
115. Khorsandi D P S, Gaslain Y. P159 Human uterine cervix-on-a-chip: establishing the first in vitro model to study the development of cervical carcinoma and human papiloma virus mechanism of action. International Journal of Gynecologic Cancer. 2019.
116. Mancini V, Pensabene V. Organs-On-Chip Models of the Female Reproductive System. Bioengineering (Basel). 2019; 6(4).
117. Xiao S, Coppeta J R, Rogers H B, et al. A microfluidic culture model of the human reproductive tract and 28-day menstrual cycle. Nat Commun. 2017; 8:14584.
118. Jagadeesan S, Workman M J, Herland A, Svendsen C N, Vatine G D. Generation of a Human iPSC-Based Blood-Brain Barrier Chip. J Vis Exp. 2020(157).
119. van der Helm M W, van der Meer A D, Eijkel J C, van den Berg A, Segerink LI. Microfluidic organ-on-chip technology for blood-brain barrier research. Tissue Barriers. 2016; 4(1):e1142493.
120. Vatine G D, Barrile R, Workman M J, et al. Human iPSC-Derived Blood-Brain Barrier Chips Enable Disease Modeling and Personalized Medicine Applications. Cell Stem Cell. 2019; 24(6):995-1005.e1006.
121. Norwitz E R, Bonney E A, Snegovskikh V V, et al. Molecular Regulation of Parturition: The Role of the Decidual Clock. Cold Spring Harb Perspect Med. 2015; 5(11).
122. Ng S W, Norwitz G A, Pavlicev M, Tilburgs T, Simon C, Norwitz E R. Endometrial Decidualization: The Primary Driver of Pregnancy Health. Int J Mol Sci. 2020; 21(11).
123. Pu Y, Gingrich J, Veiga-Lopez A. A 3-dimensional microfluidic platform for modeling human extravillous trophoblast invasion and toxicological screening. Lab Chip. 2021; 21(3):546-557.
124. Fuhler G M. The immune system and microbiome in pregnancy. Best Pract Res Clin Gastroenterol. 2020; 44-45:101671.
125. Smith S B, Ravel J. The vaginal microbiota, host defence and reproductive physiology. J Physiol. 2017; 595(2):451-463.
126. Marangoni A, Laghi L, Zagonari S, et al. New Insights into Vaginal Environment During Pregnancy. Front Mol Biosci. 2021; 8:656844.
127. Anton L, Sierra L J, DeVine A, et al. Common Cervicovaginal Microbial Supernatants Alter Cervical Epithelial Function: Mechanisms by Which Lactobacillus crispatus Contributes to Cervical Health. Front Microbiol. 2018; 9:2181.
128. Elovitz M A, Gajer P, Riis V, et al. Cervicovaginal microbiota and local immune response modulate the risk of spontaneous preterm delivery. Nat Commun. 2019; 10(1):1305.
129. Mor G. Inflammation and pregnancy: the role of toll-like receptors in trophoblast-immune interaction. Ann N Y Acad Sci. 2008; 1127:121-128.
130. Mor G, Cardenas I. The immune system in pregnancy: a unique complexity. Am J Reprod Immunol. 2010; 63(6):425-433.
131. Mor G, Kwon J Y. Trophoblast-microbiome interaction: a new paradigm on immune regulation. Am J Obstet Gynecol. 2015; 213(4 Suppl):S131-137.
132. O'Leary P, Boyne P, Flett P, Beilby J, James I. Longitudinal assessment of changes in reproductive hormones during normal pregnancy. Clin Chem. 1991; 37(5):667-672.
133. Carranza-Lira S, Hernandez F, Sanchez M, Murrieta S, Hernandez A, Sandoval C. Prolactin secretion in molar and normal pregnancy. Int J Gynaecol Obstet. 1998; 60(2):137-141.
134. Richardson L S, Taylor R N, Menon R. Reversible EMT and MET mediate amnion remodeling during pregnancy and labor. Sci Signal. 2020; 13(618).
135. Mogami H, Hari Kishore A, Akgul Y, Word RA. Healing of Preterm Ruptured Fetal Membranes. Scientific reports. 2017; 7(1):13139.
136. Gupta P, Singh M P, Goyal K. Diversity of Vaginal Microbiome in Pregnancy: Deciphering the Obscurity. Front Public Health. 2020; 8:326.
137. Ravel J, Gajer P, Abdo Z, et al. Vaginal microbiome of reproductive-age women. Proc Natl Acad Sci USA. 2011; 108 Suppl 1(Suppl 1):4680-4687.
138. Lei M, Menon R, Manteiga S, et al. Environmental Chemical Diethylhexyl Phthalate Alters Intestinal Microbiota Community Structure and Metabolite Profile in Mice. mSystems. 2019; 4(6).
139. Krishnan S, Ding Y, Saeidi N, et al. Gut Microbiota-Derived Tryptophan Metabolites Modulate Inflammatory Response in Hepatocytes and Macrophages. Cell Rep. 2019; 28(12):3285.
140. Jin U H, Lee S O, Sridharan G, et al. Microbiome-derived tryptophan metabolites and their aryl hydrocarbon receptor-dependent agonist and antagonist activities. Mol Pharmacol. 2014; 85(5):777-788.
141. Sridharan G V, Choi K, Klemashevich C, et al. Prediction and quantification of bioactive microbiota metabolites in the mouse gut. Nat Commun. 2014; 5:5492.
142. Kshirsagar S K, Alam S M, Jasti S, et al. Immunomodulatory molecules are released from the first trimester and term placenta via exosomes. Placenta. 2012; 33(12):982-990.
143. Negishi Y, Takahashi H, Kuwabara Y, Takeshita T. Innate immune cells in reproduction. J Obstet Gynaecol Res. 2018; 44(11):2025-2036.
144. Gomez-Lopez N, StLouis D, Lehr M A, Sanchez-Rodriguez E N, Arenas-Hernandez M. Immune cells in term and preterm labor. Cell Mol Immunol. 2014; 11(6):571-581.
145. Lash G E. Molecular Cross-Talk at the Feto-Maternal Interface. Cold Spring Harb Perspect Med. 2015; 5(12).
146. Liu S, Diao L, Huang C, Li Y, Zeng Y, Kwak-Kim J Y H. The role of decidual immune cells on human pregnancy. J Reprod Immunol. 2017; 124:44-53.
147. Schliefsteiner C, Peinhaupt M, Kopp S, et al. Human Placental Hofbauer Cells Maintain an Anti-inflammatory M2 Phenotype despite the Presence of Gestational Diabetes Mellitus. Front Immunol. 2017; 8:888.
148. Erlebacher A. Immunology of the maternal-fetal interface. Annu Rev Immunol. 2013; 31:387-411.
149. Nancy P, Erlebacher A. [Epigenetic repression of chemokine expression at the maternal-fetal interface as a mechanism of feto-maternal tolerance]. Med Sci (Paris). 2012; 28(12):1037-1039.
150. Nancy P, Erlebacher A. T cell behavior at the maternal-fetal interface. Int J Dev Biol. 2014; 58(2-4):189-198.
151. Gomez-Lopez N, Vega-Sanchez R, Castillo-Castrejon M, Romero R, Cubeiro-Arreola K, Vadillo-Ortega F. Evidence for a role for the adaptive immune response in human term parturition. Am J Reprod Immunol. 2013; 69(3):212-230.
152. St Louis D, Romero R, Plazyo O, et al. Invariant NKT Cell Activation Induces Late Preterm Birth That Is Attenuated by Rosiglitazone. J Immunol. 2016; 196(3):1044-1059.
153. Sayama S, Nagamatsu T, Schust D J, et al. Human decidual macrophages suppress IFN-gamma production by T cells through costimulatory B7-H1:PD-1 signaling in early pregnancy. J Reprod Immunol. 2013; 100(2):109-117.
154. Song D, Shi Y. Immune system modifications and feto-maternal immune tolerance. Chin Med J (Engl). 2014; 127(17):3171-3180.
155. Goldenberg R L, Culhane J F, lams J D, Romero R. Epidemiology and causes of preterm birth. Lancet. 2008; 371(9606):75-84.
156. Dudley D J. Immunoendocrinology of preterm labor: the link between corticotropin-releasing hormone and inflammation. Am J Obstet Gynecol. 1999; 180(1 Pt 3):S251-256.
157. Romero R, Dey S K, Fisher S J. Preterm labor: one syndrome, many causes. Science. 2014; 345(6198):760-765.
158. Gomez-Lopez N, Vadillo-Perez L, Hernandez-Carbajal A, Godines-Enriquez M, Olson D M, Vadillo-Ortega F. Specific inflammatory microenvironments in the zones of the fetal membranes at term delivery. Am J Obstet Gynecol. 2011; 205(3):235-224.
159. Romero R, Gotsch F, Pineles B, Kusanovic J P. Inflammation in pregnancy: its roles in reproductive physiology, obstetrical complications, and fetal injury. Nutr Rev. 2007; 65(12 Pt 2):S194-S202.
160. Bukowski R, Sadovsky Y, Goodarzi H, et al. Onset of human preterm and term birth is related to unique inflammatory transcriptome profiles at the maternal fetal interface. PeerJ. 2017; 5:e3685.
161. Rinaldi S F, Makieva S, Saunders P T, Rossi A G, Norman J E. Immune cell and transcriptomic analysis of the human decidua in term and preterm parturition. Mol Hum Reprod. 2017; 23(10):708-724.
162. Xu Y, Romero R, Miller D, et al. Innate lymphoid cells at the human maternal-fetal interface in spontaneous preterm labor. Am J Reprod Immunol. 2018; 79(6):e12820.
163. Hamilton S, Oomomian Y, Stephen G, et al. Macrophages infiltrate the human and rat decidua during term and preterm labor: evidence that decidual inflammation precedes labor. Biol Reprod. 2012; 86(2):39.
164. Peltier M R. Immunology of term and preterm labor. Reprod Biol Endocrinol. 2003; 1:122.
165. Zhang J, Shynlova O, Sabra S, Bang A, Briollais L, Lye S J. Immunophenotyping and activation status of maternal peripheral blood leukocytes during pregnancy and labour, both term and preterm. J Cell Mol Med. 2017; 21(10):2386-2402.
166. Edey L F, O'Dea K P, Herbert B R, et al. The Local and Systemic Immune Response to Intrauterine LPS in the Prepartum Mouse. Biol Reprod. 2016; 95(6):125.
167. Osman I, Young A, Jordan F, Greer I A, Norman J E. Leukocyte density and proinflammatory mediator expression in regional human fetal membranes and decidua before and during labor at term. J Soc Gynecol Investig. 2006; 13(2):97-103.
168. Cappelletti M, Presicce P, Lawson M J, et al. Type I interferons regulate susceptibility to inflammation-induced preterm birth. JCI Insight. 2017; 2(5):e91288.
169. Boonkasidecha S, Kannan P S, Kallapur S G, Jobe A H, Kemp M W. Fetal skin as a pro-inflammatory organ: Evidence from a primate model of chorioamnionitis. PLoS One. 2017; 12(9):e0184938.
170. Farine T, Parsons M, Lye S, Shynlova O. Isolation of Primary Human Decidual Cells from the Fetal Membranes of Term Placentae. J Vis Exp. 2018(134).
171. Jacobs S O, Sheller-Miller S, Richardson L S, Urrabaz-Garza R, Radnaa E, Menon R. Characterizing the immune cell population in the human fetal membrane. Am J Reprod Immunol. 2021; 85(5):e13368.
172. Tang Z, Tadesse S, Norwitz E, Mor G, Abrahams V M, Guller S. Isolation of hofbauer cells from human term placentas with high yield and purity. Am J Reprod Immunol. 2011; 66(4):336-348.
173. Na Q, Chudnovets A, Liu J, et al. Placental Macrophages Demonstrate Sex-Specific Response to Intrauterine Inflammation and May Serve as a Marker of Perinatal Neuroinflammation. J Reprod Immunol. 2021; 147:103360.
174. Novak C M, Ozen M, Burd I. Perinatal Brain Injury: Mechanisms, Prevention, and Outcomes. Clin Perinatol. 2018; 45(2):357-375.
175. Burd I, Balakrishnan B, Kannan S. Models of fetal brain injury, intrauterine inflammation, and preterm birth. Am J Reprod Immunol. 2012; 67(4):287-294.
176. Elovitz M A, Brown A G, Breen K, Anton L, Maubert M, Burd I. Intrauterine inflammation, insufficient to induce parturition, still evokes fetal and neonatal brain injury. Int J Dev Neurosci. 2011; 29(6):663-671.
177. Burd I, Bentz A I, Chai J, et al. Inflammation-induced preterm birth alters neuronal morphology in the mouse fetal brain. J Neurosci Res. 2010; 88(9):1872-1881.
178. Jacobs S O, Sheller-Miller S, Richardson L S, Urrabaz-Garza R, Radnaa E, Menon R. Characterizing the Immune Cell Population in the Human Fetal Membrane. Am J Reprod Immunol. 2020:e13368.
179. Gaudilliere B, Ganio E A, Tingle M, et al. Implementing Mass Cytometry at the Bedside to Study the Immunological Basis of Human Diseases: Distinctive Immune Features in Patients with a History of Term or Preterm Birth. Cytometry A. 2015; 87(9):817-829.
180. Ornatsky O, Bandura D, Baranov V, Nitz M, Winnik M A, Tanner S. Highly multiparametric analysis by mass cytometry. Journal of immunological methods. 2010; 361(1-2):1-20.
181. Tanner S D, Bandura D R, Ornatsky O, Baranov V I, Nitz M, Winnik M A. Flow cytometer with mass spectrometer detection for massively multiplexed single-cell biomarker assay. Pure Appl Chem. 2008; 80(12):14.
182. Bendall S C, Simonds E F, Qiu P, et al. Single-cell mass cytometry of differential immune and drug responses across a human hematopoietic continuum. Science. 2011; 332(6030):687-696.
183. O'Gorman W E, Hsieh E W, Savig E S, et al. Single-cell systems-level analysis of human Toll-like receptor activation defines a chemokine signature in patients with systemic lupus erythematosus. The Journal of allergy and clinical immunology. 2015; 136(5):1326-1336.
184. Qiu P, Simonds E F, Bendall S C, et al. Extracting a cellular hierarchy from high-dimensional cytometry data with SPADE. Nature biotechnology. 2011; 29(10):886-891.
185. Smith R, Mesiano S, McGrath S. Hormone trajectories leading to human birth. Regul Pept. 2002; 108(2-3):159-164.
186. Wisniewski J R, Hein M Y, Cox J, Mann M. A “proteomic ruler” for protein copy number and concentration estimation without spike-in standards. Mol Cell Proteomics. 2014; 13(12):3497-3506.
187. Langfelder P, Horvath S. WGCNA: an R package for weighted correlation network analysis. BMC Bioinformatics. 2008; 9:559.
188. Kramer A, Green J, Pollard J, Jr., Tugendreich S. Causal analysis approaches in Ingenuity Pathway Analysis. Bioinformatics. 2014; 30(4):523-530.
189. Shannon P, Markiel A, Ozier O, et al. Cytoscape: a software environment for integrated models of biomolecular interaction networks. Genome Res. 2003; 13(11):2498-2504.
190. Wu S P, Anderson M L, Wang T, et al. Dynamic transcriptome, accessible genome, and PGR cistrome profiles in the human myometrium. Faseb j. 2020; 34(2):2252-2268.
191. Niu C, Cheng C, Liu Y, Huang S, Fu Y, Li P. Transcriptome Profiling Analysis of Bovine Vaginal Epithelial Cell Response to an Isolated Lactobacillus Strain. mSystems. 2019; 4(5).
192. Harlow K, Suarez-Trujillo A, Hedrick V, et al. Temporal analysis of vaginal proteome reveals developmental changes in lower reproductive tract of gilts across the first two weeks postnatal. Scientific reports. 2019; 9(1):13241.
193. Femandez-Hermida Y, Grande G, Menarguez M, Astorri A L, Azagra R. Proteomic Markers in Cervical Mucus. Protein Pept Lett. 2018; 25(5):463-471.
194. Dasari S, Pereira L, Reddy A P, et al. Comprehensive proteomic analysis of human cervical-vaginal fluid. J Proteome Res. 2007; 6(4):1258-1268.
195. Buhimschi C S, Rosenberg V A, Dulay A T, et al. Multidimensional system biology: genetic markers and proteomic biomarkers of adverse pregnancy outcome in preterm birth. Am J Perinatol. 2008; 25(3):175-187.
196. Parry S, Leite R, Esplin M S, et al. Cervicovaginal fluid proteomic analysis to identify potential biomarkers for preterm birth. Am J Obstet Gynecol. 2020; 222(5):493 e491-493 e413.
197. Krjutskov K, Katayama S, Saare M, et al. Single-cell transcriptome analysis of endometrial tissue. Hum Reprod. 2016; 31(4):844-853.
198. Sakabe N J, Aneas I, Knoblauch N, et al. Transcriptome and regulatory maps of decidua-derived stromal cells inform gene discovery in preterm birth. Sci Adv. 2020; 6(49).
199. Stanfield Z, Lai P F, Lei K, et al. Myometrial Transcriptional Signatures of Human Parturition. Front Genet. 2019; 10:185.
200. Mittal P, Romero R, Tarca A L, et al. Characterization of the myometrial transcriptome and biological pathways of spontaneous human labor at term. Journal of perinatal medicine. 2010; 38(6):617-643.
201. Pavlicev M, Wagner G P, Chavan A R, et al. Single-cell transcriptomics of the human placenta: inferring the cell communication network of the maternal-fetal interface. Genome Res. 2017; 27(3):349-361.
202. Michelsen T M, Henriksen T, Reinhold D, Powell T L, Jansson T. The human placental proteome secreted into the maternal and fetal circulations in normal pregnancy based on 4-vessel sampling. FASEB J. 2019; 33(2):2944-2956.
203. Khorami Sarvestani S, Shojaeian S, Vanaki N, et al. Proteome profiling of human placenta reveals developmental stage-dependent alterations in protein signature. Clin Proteomics. 2021; 18(1):18.
204. Kasiviswanathan D, Chinnasamy Perumal R, Bhuvaneswari S, et al. Interactome of miRNAs and transcriptome of human umbilical cord endothelial cells exposed to short-term simulated microgravity. NPJ Microgravity. 2020; 6:18.
205. Park S J, Yoon W G, Song J S, et al. Proteome analysis of human amnion and amniotic fluid by two-dimensional electrophoresis and matrix-assisted laser desorption/ionization time-of-flight mass spectrometry. Proteomics. 2006; 6(1):349-363.
206. Tarca A L, Romero R, Pique-Regi R, et al. Amniotic fluid cell-free transcriptome: a glimpse into fetal development and placental cellular dynamics during normal pregnancy. Bmc Med Genomics. 2020; 13(1):25.
207. Darmanis S, Sloan S A, Zhang Y, et al. A survey of human brain transcriptome diversity at the single cell level. Proc Natl Acad Sci USA. 2015; 112(23):7285-7290.
208. Nascimento J M, Saia-Cereda V M, Sartore R C, et al. Human Cerebral Organoids and Fetal Brain Tissue Share Proteomic Similarities. Front Cell Dev Biol. 2019; 7:303.
209. Mendelson C R. Minireview: fetal-maternal hormonal signaling in pregnancy and labor. Mol Endocrinol. 2009; 23(7):947-954.
210. Casey M L, MacDonald P C. Endocrinology of preterm birth. Clin Obstet Gynecol. 1984; 27(3):562-571.
211. Mitchell M D, Romero R J, Edwin S S, Trautman M S. Prostaglandins and parturition. Reprod Fertil Dev. 1995; 7(3):623-632.
212. Reinl E L, England S K. Fetal-to-maternal signaling to initiate parturition 1. J Clin Invest. 2015; 125(7):2569-2571.
213. Montalbano A P, Hawgood S, Mendelson C R. Mice deficient in surfactant protein A (SP-A) and SP-D or in TLR2 manifest delayed parturition and decreased expression of inflammatory and contractile genes. Endocrinology. 2013; 154(1):483-498.
214. Condon J C, Jeyasuria P, Faust J M, Mendelson C R. Surfactant protein secreted by the maturing mouse fetal lung acts as a hormone that signals the initiation of parturition. Proc Natl Acad Sci USA. 2004; 101(14):4978-4983.
215. Casey M L, Brown C E, Peters M, MacDonald P C. Endothelin levels in human amniotic fluid at mid-trimester and at term before and during spontaneous labor. J Clin Endocrinol Metab. 1993; 76(6):1647-1650.
216. Romero R, Avila C, Edwin S S, Mitchell M D. Endothelin-1,2 levels are increased in the amniotic fluid of women with preterm labor and microbial invasion of the amniotic cavity. Am J Obstet Gynecol. 1992; 166(1 Pt 1):95-99.
217. Brady R, Zaidi S I, Mayer C, Katz D M. BDNF is a target-derived survival factor for arterial baroreceptor and chemo-afferent primary sensory neurons. J Neurosci. 1999; 19(6):2131-2142.
218. Hoffman D R, Romero R, Johnston J M. Detection of platelet-activating factor in amniotic fluid of complicated pregnancies. Am J Obstet Gynecol. 1990; 162(2):525-528.
219. Richardson L, Menon R. Proliferative, Migratory, and Transition Properties Reveal Metastate of Human Amnion Cells. Am J Pathol. 2018; 188(9):2004-2015.
220. Polettini J, Richardson L S, Menon R. Oxidative stress induces senescence and sterile inflammation in murine amniotic cavity. Placenta. 2018; 63:26-31.
221. Woods J R, Jr. Reactive oxygen species and preterm premature rupture of membranes-a review. Placenta. 2001; 22 Suppl A:S38-S44.
222. MacDonald P C, Casey M L. The accumulation of prostaglandins (PG) in amniotic fluid is an aftereffect of labor and not indicative of a role for PGE2 or PGF2 alpha in the initiation of human parturition. J Clin Endocrinol Metab. 1993; 76(5):1332-1339.
223. Kaneko K, Shimizu H, Arakawa H, Ogawa Y. Pulmonary surfactant protein A in sera for assessing neonatal lung maturation. Early Hum Dev. 2001; 62(1):11-21.
224. Lonergan M, Aponso D, Marvin K W, et al. Tumor necrosis factor-related apoptosis-inducing ligand (TRAIL), TRAIL receptors, and the soluble receptor osteoprotegerin in human gestational membranes and amniotic fluid during pregnancy and labor at term and preterm. J Clin Endocrinol Metab. 2003; 88(8):3835-3844.
225. Romero R, Chaemsaithong P, Docheva N, et al. Clinical chorioamnionitis at term IV: the maternal plasma cytokine profile. Journal of perinatal medicine. 2016; 44(1):77-98.
226. Romero R, Mazor M, Brandt F, et al. Interleukin-1 alpha and interleukin-1 beta in preterm and term human parturition. Am J Reprod Immunol. 1992; 27(3-4):117-123.
227. Figueroa R, Garry D, Elimian A, Patel K, Sehgal P B, Tejani N. Evaluation of amniotic fluid cytokines in preterm labor and intact membranes. J Matern Fetal Neonatal Med. 2005; 18(4):241-247.
228. Dixon C L, Richardson L, Sheller-Miller S, Saade G, Menon R. A distinct mechanism of senescence activation in amnion epithelial cells by infection, inflammation, and oxidative stress. Am J Reprod Immunol. 2018; 79(3).
229. Uvnas-Moberg K, Ekstrom-Bergstrom A, Berg M, et al. Maternal plasma levels of oxytocin during physiological childbirth—a systematic review with implications for uterine contractions and central actions of oxytocin. BMC Pregnancy Childbirth. 2019; 19(1):285.
230. Bakker R, Pierce S, Myers D. The role of prostaglandins E1 and E2, dinoprostone, and misoprostol in cervical ripening and the induction of labor: a mechanistic approach. Arch Gynecol Obstet. 2017; 296(2):167-179.
231. Pierce S, Bakker R, Myers D A, Edwards R K. Clinical Insights for Cervical Ripening and Labor Induction Using Prostaglandins. AJP Rep. 2018; 8(4):e307-e314.
232. Richardson L, Jeong S, Kim S, Han A, Menon R. Amnion membrane organ-on-chip: an innovative approach to study cellular interactions. FASEB J. 2019; 33(8):8945-8960.
233. Matzke M M, Waters K M, Metz T O, et al. Improved quality control processing of peptide-centric LC-MS proteomics data. Bioinformatics. 2011; 27(20):2866-2872.
234. Webb-Robertson B J, McCue L A, Waters K M, et al. Combined statistical analyses of peptide intensities and peptide occurrences improves identification of significant peptides from MS-based proteomics data. J Proteome Res. 2010; 9(11):5748-5756.
235. Webb-Robertson B J, Matzke M M, Jacobs J M, Pounds J G, Waters K M. A statistical selection strategy for normalization procedures in LC-MS proteomics experiments through dataset-dependent ranking of normalization scaling factors. Proteomics. 2011; 11(24):4736-4741.
236. Dunnett C W. A Multiple Comparison Procedure for Comparing Several Treatments with a Control. Journal of the American Statistical Association. 1955; 50(272):1096-1121.
237. Bender R, Lange S. Adjusting for multiple testing—when and how? J Clin Epidemiol. 2001; 54(4):343-349.
238. Storey J D. The Positive False Discovery Rate: A Bayesian Interpretation and the q-Value The Annals of Statistics. 2003; 31(6):2013-2035.
239. Webb-Robertson B J, Matzke M M, Metz T O, et al. Sequential projection pursuit principal component analysis—dealing with missing data associated with new-omics technologies. Biotechniques. 2013; 54(3):165-168.
240. UniProt C. UniProt: a worldwide hub of protein knowledge. Nucleic Acids Res. 2019; 47(D1):D506-D515.
241. Du J, Yuan Z, Ma Z, Song J, Xie X, Chen Y. KEGG-PATH: Kyoto encyclopedia of genes and genomes-based pathway analysis using a path analysis model. Mol Biosyst. 2014; 10(9):2441-2447.
242. Kanehisa M, Sato Y, Furumichi M, Morishima K, Tanabe M. New approach for understanding genome variations in KEGG. Nucleic Acids Res. 2019; 47(D1):D590-D595.
243. Li M, Brokaw A, Furuta A M, et al. Non-human Primate Models to Investigate Mechanisms of Infection-Associated Fetal and Pediatric Injury, Teratogenesis and Stillbirth. Front Genet. 2021; 12:680342.
244. Adams Waldorf K M, Rubens C E, Gravett M G. Use of nonhuman primate models to investigate mechanisms of infection-associated preterm birth. BJOG. 2011; 118(2):136-144.
245. US Department of Health and Human Services. Nonhuman Primate Tissue Bank Handbook. National Institute on Aging website. Accessed Aug. 27, 2021. https://www.nia.nih.gov/research/dab/nonhuman-primate-tissue-bank-handbook

1. A 3D microfluidic device mimicking an anatomy and physiology of a pregnancy comprising:
a body defining a central cavity, an inlet aperture and an outlet aperture, wherein the apertures are in fluid communication with the central cavity of the body, wherein the central cavity comprises decidual cells;
a first plurality of chambers in fluid communication with the central cavity, wherein the first plurality of chambers comprise cells of fetal origin;
a second plurality of chambers in fluid communication with the central cavity opposite the first plurality of chambers, wherein the second plurality of chambers comprise cells of maternal origin; and
a third plurality of chamber in fluid communication with the central cavity, wherein the third plurality of chambers comprise cells of the myometrium;
wherein the integration of inputs and outputs from the central chamber, the first, second, and third pluralities of chambers mimics the anatomy and physiology of pregnancy.

2. The microfluidic device of claim 1, wherein the inlet aperture and the outlet aperture allow for injection of at least one of fluid, gas and solid material within and through the device.

3. The microfluidic device of claim 1, wherein the device is configured to be filled with cellular components in the first channel, second channel, top cavity and bottom cavity.

4. The microfluidic device of claim 1, further comprising an outer surface for sealing the device to a support base.

5. The microfluidic device of claim 1, further comprising a support base, optionally a glass support base.

6. The microfluidic device of claim 1, wherein the device comprises a material selected from a group consisting of glass, silicon, polysiloxane, polydimethylsiloxane, and optically transparent polymers.

7. The microfluidic device of claim 1, wherein the decidual cells form a decidual-placental interface, comprising one more cells selected from: decidua (DEC), syncytiotrophoblasts (STB), cytotrophoblast (CTB), and human umbilical vein endothelial cells (HUVEC).

8. The microfluidic device of claim 1, wherein the fetal cells form Fetal membrane-decidual interface that comprise one or more cells selected from (4 cell types)—decidua (DEC), chorion trophoblast cells (CTC), extracellular matrix mesenchymal cells (MC), and amnion epithelial cells (AEC).

9. The microfluidic device of claim 1, wherein the maternal cells for a Vagina-Cervix and comprise one or more cells selected from: cervical epithelial (ECTO and ENDO) and stromal cells (STR).

10. The microfluidic device of claim 1, wherein the myometrium cells are selected from myometrial smooth muscle cells (MYO).

11. The microfluidic device of claim 1, further comprising a fourth chamber comprising fetal brain cells (FB).

12. A lab-on-a-chip comprising a 3D microfluidic device mimicking an anatomy and physiology of a pregnancy comprising:
a body defining a central cavity, an inlet aperture and an outlet aperture, wherein the apertures are in fluid communication with the central cavity of the body, wherein the central cavity comprises cells of the decidua:
a first plurality of chambers in fluid communication with the central cavity, wherein the first plurality of chambers comprise cells of fetal origin;
a second plurality of chambers in fluid communication with the central cavity opposite the first plurality of chambers, wherein the second plurality of chambers comprise cells of maternal origin; and
a third plurality of chamber in fluid communication with the central cavity, wherein the third plurality of chambers comprise cells of the myometrium;
wherein the integration of inputs and outputs from the central chamber, the first, second, and third pluralities of chambers mimics the anatomy and physiology of pregnancy.

13. The lab-on-a-chip of claim 12, wherein the inlet aperture and the outlet aperture allow for injection of at least one of fluid, gas and solid material within and through the device.

14. The lab-on-a-chip of claim 12, wherein the device comprises a material selected from a group consisting of glass, silicon, polysiloxane and optically transparent polymers, optionally wherein the polysiloxane is polydimethylsiloxane (PDMS).

15. The lab-on-a-chip of claim 12, wherein the width of the first channel is smaller than the width of the second channel.

16. The lab-on-a-chip of claim 12, further comprising hydrogels contained separately within the first channel, second channel, bottom cavity and top cavity of the device.

17. The lab-on-a-chip of claim 16, wherein the second channel comprises a porous hydrogel of Type I collagen.

18. The lab-on-a-chip of claim 16, wherein the hydrogel of the top cavity comprises viable B cells, and the hydrogel of the bottom cavity comprises viable T cells.

19. The lab-on-chip of claim 16, wherein the top cavity further comprises chemokine CXCL13.

20. The lab-on-chip of claim 16, wherein the bottom cavity further comprises a chemokine selected from the group consisting of CXCL12, CCL21, and CCL19, or a combination thereof.

21. A kit comprising a lab-on-a-chip comprising:
a 3D microfluidic device mimicking an anatomy and physiology of a pregnancy comprising:
a body defining a central cavity, an inlet aperture and an outlet aperture, wherein the apertures are in fluid communication with the central cavity of the body, wherein the central cavity comprises cells of the decidua:
a first plurality of chambers in fluid communication with the central cavity, wherein the first plurality of chambers comprise cells of fetal origin;
a second plurality of chambers in fluid communication with the central cavity opposite the first plurality of chambers, wherein the second plurality of chambers comprise cells of maternal origin;
a third plurality of chamber in fluid communication with the central cavity, wherein the third plurality of chambers comprise cells of the myometrium;
wherein the integration of inputs and outputs from the central chamber, the first, second, and third pluralities of chambers mimics the anatomy and physiology of pregnancy; and
one or more vials comprising the one or more cells of fetal and non-fetal origin.
